SEQUENCE LISTING (14 sequences)

SEQ ID NO: 1 - DNA, 20 bp, Artificial Sequence
  synthetic construct
acccagaagactgtggatgg

SEQ ID NO: 2 - DNA, 20 bp, Artificial Sequence
  synthetic construct
cacattgggggtaggaacac

SEQ ID NO: 3 - DNA, 20 bp, Artificial Sequence
  synthetic construct
gcaacgggaagattctgaag

SEQ ID NO: 4 - DNA, 20 bp, Artificial Sequence
  synthetic construct
tgacaaacttctgcctgacg

SEQ ID NO: 5 - DNA, 20 bp, Artificial Sequence
  synthetic construct
agttgccttcttgggactga

SEQ ID NO: 6 - DNA, 20 bp, Artificial Sequence
  synthetic construct
tccaggatttcccagagaac

SEQ ID NO: 7 - DNA, 24 bp, Artificial Sequence
  synthetic construct
cacactcagatcatcttctcaaaa

SEQ ID NO: 8 - DNA, 24 bp, Artificial Sequence
  synthetic construct
gcaatgactctaaagtagacctgc

SEQ ID NO: 9 - DNA, 20 bp, Artificial Sequence
  synthetic construct
acccagaagactgtggatgg

SEQ ID NO: 10 - DNA, 20 bp, Artificial Sequence
  synthetic construct
cacattgggggtaggaacac

SEQ ID NO: 11 - DNA, 20 bp, Artificial Sequence
  synthetic construct
tcccagaagactgtggatgg

SEQ ID NO: 12 - DNA, 19 bp, Artificial Sequence
  synthetic construct
gcaatttcccaagtctctg

SEQ ID NO: 13 - DNA, 20 bp, Artificial Sequence
  synthetic construct
acacacaggtgggacacaaa

SEQ ID NO: 14 - DNA, 20 bp, Artificial Sequence
  synthetic construct
ctggggtggtctcgatttta

(19) United States
(12) Patent Application Publication
(10) Pub. No.: US 2016/0136214 A1
(43) Pub. Date: May 19, 2016

(54) SESAME SEED OIL AQUEOUS EXTRACTS AND METHODS OF MAKING AND USING THEREOF

(71) Applicant: University of Central Florida Research Foundation, Inc., Orlando, FL (US)

(72) Inventors: Sampath Parthasarathy, Orlando, FL (US); Aluganti Narashimhulu Chandrakala, Orlando, FL (US)

(21) Appl. No.: 14/937,475

(22) Filed: Nov. 10, 2015

Related U.S. Application Data
(60) Provisional application No. 62/077,575, filed on Nov. 10, 2014.

(51) Int. Cl. A61K 36/185; A23L 2/52; A23L 2/60; A61K 9/00

(52) U.S. Cl. CPC A61K 36/185; A61K 9/0056; A61K 9/0095; A23L 1/3002; A23L 2/52; A23L 2/60; A23L 1/30; A23K 1/1643; A23K 1/1646

(57) ABSTRACT

Described are compositions and methods for extracting non-lipid, aqueous components from sesame seed oil. Such compositions are useful for methods of preventing, treating and inhibiting the effects of inflammation in the body. The methods disclosed involve the use of sesame seed oil aqueous extracts for the treatment and prevention of inflammatory related conditions including atherosclerosis, arthritis, diabetes, mental conditions and vision impairment.

### CROSS REFERENCE TO RELATED APPLICATIONS

This application claims the benefit of priority to U.S. Provisional Application No. 62/077,575, filed Nov. 10, 2014, which is incorporated by reference herein in its entirety.

### STATEMENT OF GOVERNMENT SUPPORT

This invention was made with government support under Grant No. R01 AT004106 awarded by the National Institutes of Health. The government has certain rights in this invention.

### FIELD

The disclosed subject matter relates to methods for extracting non-lipid, aqueous components associated with sesame seed oil and incorporating a sesame seed oil aqueous extract into compositions, for example, to treat or prevent inflammation and related pathologies such as atherosclerosis in humans and animals. More particularly, the disclosed subject matter relates to compositions comprising sesame seed oil aqueous extracts having anti-inflammatory properties.

### BACKGROUND

Sesame seeds are known to be potent, nutrient-dense foods praised not only for their nutritional content in seed form, but also highly valued for their rancid-resistant oil. Sesame seeds have a strong nutritional profile, supporting their capabilities in fighting, preventing, and reversing illness and disease. The seeds are especially high in copper, manganese, calcium, and magnesium and also contain vitamins, minerals, and other nutrients such as manganese, copper, calcium, iron, magnesium, tryptophan, zinc, fiber, thiamin, vitamin B6, phosphorous and protein. They also contain many “antioxidants”, such as sesamol, sesamin etc.

The health benefits of sesame seeds and sesame seed oil are plentiful. As sesame seeds are full of zinc, an essential mineral for producing collagen and giving skin more elasticity, sesame seed oil is associated with promoting healthy skin, and alleviating the repair of damaged tissues. In addition, because sesame seeds offer close to five grams of protein per ounce, they are desirable for high-protein vegetarian diets. Some studies have shown that sesame seed oil is also good for boosting oral health.

As a result of the high magnesium and other nutrient content, sesame seeds, and especially sesame seed oil, have been shown to combat diabetes, reduce blood pressure and promote heart health. Additional studies have demonstrated that sesamol, a compound found in sesame seeds and sesame seed oil, can protect against DNA damage caused by radiation.

Sesame seeds contain an anti-cancer compound called phytate, which in addition to the magnesium also present in the sesame, can harness anti-cancer properties. The zinc content of sesame is also beneficial for boosting bone mineral density and bone health a whole. Additional health benefits associated with sesame seeds and sesame seed oil include improving digestive health, relieving constipation, and promoting respiratory health.

Given the beneficial attributes of sesame seeds and sesame seed oil, what is needed are effective methods for extracting useful and active components from sesame seeds and sesame seed oil in order to provide such components in compositions that are easy to administer, consume, and ingest. Consuming large quantities of sesame seeds or sesame seed oil in order to obtain significant physiologically positive benefits is not routinely practical or feasible. Besides, consuming large quantities of sesame oil also brings-in large amounts of fat calories which is undesirable. Accordingly, what is needed is a method by which beneficial extracts can be efficiently obtained and incorporated into compositions such as beverages and foods in order to achieve optimal health benefits. What is also needed is a method for incorporating desired extracts from sesame seeds and sesame seed oil into compositions for mitigating and preventing complications and negative effects of inflammation. The compositions and methods disclosed herein address these and other needs.

### SUMMARY

Disclosed herein are compositions and methods of making and using such compositions. In a more specific aspect, disclosed herein are methods for extracting non-lipid, aqueous components of sesame seed oil and incorporating such components in compositions used to treat or prevent inflammation. Also disclosed are compositions that comprise sesame seed oil aqueous extracts that are useful for inhibiting inflammatory reactions in humans or animals, and for preventing and treating inflammation-related conditions. Further, disclosed are methods that comprise extracting non-lipid, aqueous components associated with sesame seed oil (SOAE, sesame seed oil aqueous extract), methods of using SOAE for treating or preventing inflammation-related conditions comprising providing an effective amount of a sesame seed oil aqueous extract containing composition to prevent or reduce inflammation events occurring in a human or animal.

In specific aspects, disclosed herein are dietary compositions, foods, beverages and items for consumption that comprise sesame seed oil aqueous extracts, including but not limited to SOAE. For example, disclosed herein are beverages that comprise sesame seed oil aqueous extracts.

In a specific aspect, disclosed herein are methods of treating inflammation-related conditions that comprises administering to a human or animal in need thereof an anti-inflammatory comestible composition comprising a food or beverage and a sesame seed oil aqueous extract, wherein the sesame seed oil aqueous extract is administered in an amount effective to reduce inflammation in the human or animal. The inflammation-related condition can be selected from the group consisting of atherosclerosis, Alzheimer's Disease, allergies, myopathies, leukocyte defects, cancer, cardiovascular diseases, inflammatory bowel diseases, pelvic inflammatory diseases, and inflammatory diseases in the brain.

The comestible composition can be a beverage selected from the group consisting of coffee, tea, fruit juice, vegetable juice, milk, energy drink, protein drink, soft drink, and alcoholic drink, or a sweetener composition. The composition can also be a frozen liquid or a powder. Nutraceutical, pharmaceutical, or dietary supplements can also be added to the compositions.

Also disclosed herein are methods of preparing a sesame seed oil aqueous extract is by contacting a sesame seed oil with water to form a mixture; mixing the mixture to form an aqueous layer and an oil layer; and isolating the aqueous layer, which is substantially free of lipids, thereby producing the sesame seed oil aqueous extract. The method can also comprise dehydrating the sesame seed oil aqueous extract.

In further aspects, disclosed herein are methods of preparing an anti-inflammatory comestible composition that comprises contacting a sesame seed oil with water to form a mixture; mixing the mixture to form an aqueous layer and an oil layer; isolating the aqueous layer, which is substantially free of lipids, to provide a sesame seed oil aqueous extract; and combining the sesame seed oil aqueous extract with a food or beverage. Mixing the mixture can be by shaking the mixture, stirring the mixture, bubbling a gas through the mixture, or applying ultrasound to the mixture. The aqueous layer can be filtrated to isolate the extract. The extract can also be dehydrated.

Foods and beverages comprising the disclosed sesame seed oil aqueous extract are also disclosed. They can be prepared by combining from about 0.001 mg/mL to about 1000 mg/mL of the sesame seed oil aqueous extract to the food or beverage. If dehydrated and in powder form, the sesame seed oil extract can be combined with the food or beverage at from about 0.001 mg to about 1000 mg, w/w. Examples of beverages that ca be used are coffee, tea, fruit juice, vegetable juice, milk, energy drink, protein drink, soft drink, and alcoholic drink.

Also disclosed are an anti-inflammatory comestible compositions that comprise a food or beverage and a sesame seed oil aqueous extract in an amount effective to reduce inflammation in a human or animal. The sesame seed oil aqueous extract is prepared by contacting a sesame seed oil with water to form a mixture; mixing the mixture to form an aqueous layer and an oil layer; and isolating the aqueous layer, which is substantially free of lipids, thereby producing the sesame seed oil aqueous extract.

Currently consumption of at least 2 liters of beverages per day is recommended to keep the body hydrated and to maintain proper kidney function. People drink a variety of beverages from hot beverages such as coffee and tea to soda and juices. While these can provide hydration, the presence of high amounts of sugars, artificial chemical ingredients, caffeine, and others components are of health concern. Furthermore, drinks that have very high amounts of sugars are promoted as high-energy supplements, causing additional concern as high sugar promotes inflammation. Despite these problems, the availability of beverages containing sugar as well as other unhealthy ingredients continues to increase and consumption rates of such beverages has also increased exponentially during the past decade. Inflammation has now been recognized to be a major player in many chronic diseases. Disclosed herein are beverages or items of consumption comprising sesame seed oil aqueous extract that counterbalances the harmful effects of inflammation-causing ingredients (e.g. high sugar intake).

Many edible oils have potent biological properties, for example anti-inflammatory, antioxidant, or anti-atherosclerotic effects. These oils have both fat and non-fat components associated with them. For example, extra-virgin olive oil, sesame seed oil, and many others comprise numerous non-fat components (e.g. tocopherols, polyphenols etc.). Many of these have been reported to have beneficial effects. However, the need to consume large amounts of oils to derive benefits from these has precluded their use in populations who need to restrict their fat calories. In addition, people tend to use edible oils based on their preference, cost, and other factors.

Positive anti-atherosclerotic effects of sesame seed oil (SO) have been demonstrated in mouse models of atherosclerosis and a detailed analysis has shown that these effects also include potent anti-inflammatory effects. Nevertheless, consuming large amounts of SO is not practical as it would bring unwanted additional fat calories, and accordingly in an effort to satisfy this need, disclosed herein are simple methods of extracting beneficial non-fat sesame seed oil aqueous extracts from SO.

Disclosed herein are methods comprising freeze-drying and reconstituting desirable sesame seed oil aqueous extracts to prepare compositions comprising a sesame seed oil aqueous extract. As discussed in the Examples, laboratory studies conducted using the sesame seed oil aqueous extracts, showed that the sesame seed oil aqueous extracts demonstrated potent anti-inflammatory effects. The sesame seed oil aqueous extracts also inhibited bacterial lipopolysaccharide-induced inflammatory stress in a mouse model. In addition, the sesame seed oil aqueous extracts also significantly inhibited atherosclerosis in a mouse model of atherosclerosis.

Disclosed and included herein are compositions which can be foods, beverages or compositions for consumption by humans or animals, which comprise SOAE including, but not limited to, beverages (i.e. fruit juice, coffee, tea, energy drinks, sodas), food stuffs (i.e. bread, dairy products, desserts, savory items, cereals etc.), oral products (i.e. toothpaste, mouthwash, coated dental floss etc.) and wound care articles (i.e. personal adhesive bandages, bandages, gauze pads, medical tape, swabs, towelettes, wipes etc.). Disclosed and included herein are compositions for personal use and other methods for delivering beneficial components of sesame seed oil aqueous extracts to a subject, including but not limited to wound healing devices (i.e. personal adhesive bandages, bandages, gauze pads, medical tape, swabs, towelettes, wipes, suture kits, wound adhesives, gels, topical ointments, salves etc.). In certain aspects, the sesame seed oil aqueous extracts can be delivered via intravenous infusion, or transdermal patches. Delivery of sesame seed oil aqueous extracts, including delivery of SOAE extracts in such a manner can alleviate negative symptoms associated with inflammation. The disclosed subject matter contemplates administration of an effective amount of a composition comprising a sesame seed oil aqueous extract, which can be delivered by any known route of administration to a human or animal. Pharmaceutical compositions comprising a sesame seed oil aqueous extract, along with known pharmaceutical excipients or other components, is disclosed herein.

Disclosed herein are beverage compositions, comprising, an aqueous extract of sesame seed oil in a water-based liquid, wherein the sesame seed oil aqueous extract is made by a process comprising, adding a portion of sesame seed oil to a water-based liquid to form a mixture; shaking the mixture; filtering the mixture to separate at least a portion of the lipids to form a filtrate substantially free of lipids; wherein the filtrate comprises a sesame seed oil aqueous extract. Such beverage compositions can comprise any beverage, including but not limited to beverages wherein the water-based liquid comprises fruit juice, coffee, tea or soda.

Disclosed herein is a method for making sesame seed oil aqueous extracts, comprising admixing sesame seed oil with water in a 1:0.25 ratio, shaking the mixture, filtering the mixture to separate at least a portion of the lipids to form a filtrate substantially free of lipids. The extraction is repeated twice (or more) and the combined aqueous extracts are refiltered.

Disclosed herein is a method for making sesame seed oil aqueous extracts, comprising admixing sesame seed oil with water in a 1:0.25 ratio, shaking the mixture, filtering the mixture to separate at least a portion of the lipids to form a filtrate substantially free of lipids, and further comprising, dehydrating the filtrate substantially free of lipids to form a sesame seed oil aqueous extract powder.

Disclosed herein is a method for making sesame seed oil aqueous extracts, comprising admixing sesame seed oil with water in a 1:0.25 ratio, shaking the mixture, filtering the mixture to separate at least a portion of the lipids to form a filtrate substantially free of lipids, wherein filtering comprises allowing the mixture to flow through a filter paper of 2-12, 3-9, or 5 μm pore size.

Disclosed herein is a method for making sesame seed oil aqueous extracts, comprising admixing sesame seed oil with water in a 1:0.25 ratio, shaking the mixture, filtering the mixture to separate at least a portion of the lipids to form a filtrate substantially free of lipids, wherein the step of filtering is repeated more than one time, with the filtrate of the previous filtering step flowing through a new filter paper of 2-12, 3-9, or 5 micron pore size.

Disclosed herein is a method for making sesame seed oil aqueous extracts comprising admixing sesame seed oil with water in a 1:0.25 ratio, shaking the mixture, filtering the mixture to separate at least a portion of the lipids to form a filtrate substantially free of lipids, wherein the shaking is performed in an opaque container.

Disclosed herein is a method for making sesame seed oil aqueous extracts, comprising admixing sesame seed oil with water in a 1:0.25 ratio, shaking the mixture, filtering the mixture to separate at least a portion of the lipids to form a filtrate substantially free of lipids, dehydrating the filtrate substantially free of lipids to form an aqueous exact of sesame seed oil powder. Disclosed herein are items of consumptions such as beverages and foods comprising aqueous exact of sesame seed oil powder.

Disclosed herein are methods of treating, ameliorating, modulating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal.

Disclosed herein are methods of treating, ameliorating, modulating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal and wherein the composition is provided to the human or animal as a component of a beverage, or other consumable composition.

Disclosed herein are methods of treating, ameliorating, modulating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal, wherein the composition is provided to the human or animal in a beverage, or other consumable composition and wherein the inflammation-related condition is atherosclerosis.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE FIGURES

The accompanying figures, which are incorporated in and constitute a part of this specification, illustrate several aspects and together with the description serve to explain the principles of the invention.

FIG. 1 is a depiction of how a SOAE is prepared and how the extract can be used for a variety of applications, such as an additive or supplement to beverages, oral products, wound care articles, or intravenous (IV) infusions.

FIG. 2A contains an absorption spectral analysis of both sesamol and SOAE between 200 nm and 300 nm. FIG. 2B is a thin layer chromatography analysis of both sesamol and SOAE. It is to be noted that the SOAE contained additional components that are distinct from sesamol.

FIGS. 3A through 3D show that SOAE inhibits LPS-induced inflammation in RAW cells. FIG. 3A and FIG. 3D contain data of IL-1α mRNA levels, FIG. 3B and FIG. 3D contain data of IL-6 mRNA levels, and FIG. 3C and FIG. 3D contain data of TNF-α mRNA levels in RAW cells treated with lipopolysaccharide (LPS) to induce inflammation and sesame seed oil aqueous extract (SOAE). Protein levels of IL-1α, IL-6, and TNF-α were determined by Western blot analysis (FIG. 3E).

FIG. 4A contains micrographs showing GFP reporter expression of HepG2-LXR cells treated with sesame seed oil aqueous extract (SOAE). FIG. 4B shows levels of luciferase activity in HepG2-LXR cells treated with sesame seed oil aqueous extract (SOAE).

FIGS. 5A through 5H show that SOAE inhibits LPS-induced inflammatory cytokines in RAW cells. FIG. 5A contains scatter plots showing relative expression levels of genes in LPS treated cells and LPS+SOAE treated RAW cells. RAW cells were pre-treated with SOAE followed by treatment with LPS. Following 24 hours of incubation, RNA was isolated. The Mouse Atherosclerosis PCR array (Qiagen) was carried out with these samples. Relative expression levels of genes in LPS treated cells (FIG. 5A, left) and LPS+SOAE treated cells (FIG. 5A, right) are plotted against a control in the Scatter Plot. Genes that were distinctly down-regulated (FIG. 5B) and up-regulated (FIG. 5C) are represented as bar diagrams. Real time PCR analysis was performed for IL-1α (FIG. 5D), IL-6 (FIG. 5E) and TNF-α (FIG. 5F). ELISA to detect mouse IL-6 (FIG. 5G) and TNF-α (FIG. 5H) secretion in to the medium was also performed. SOAE significantly attenuated the expression of LPS-induced inflammatory markers. Results are represented as mean±SE from more than three independent experiments. *P<0.05.

FIGS. 6A through 6C show SOAE inhibits LPS-induced inflammatory cytokines in mouse peritoneal macrophages. Mouse peritoneal macrophages were pre-treated with SOAE (200 μg/mL) followed by addition of 10 ng/mL LPS. Cells were incubated for 24 hours following which RNA was isolated and real time PCR analysis was performed for IL-1α (FIG. 6A), IL-6 (FIG. 6B) and TNF-α (FIG. 6C) gene expression. (n=3; * P<0.05).

FIGS. 7A through 7F show that SOAE inhibits VCAM1 and MCP-1 expression in HUVECs. HUVECs were pre-treated with SOAE followed by addition of 10 ng/mL TNF-α. Cells were incubated for 24 hours at the end of which total cellular RNA was extracted and reverse transcribed. Real time PCR analysis was performed to analyze mRNA levels of VCAM1 (FIG. 7A) and MCP-1 (FIG. 7B). PCR products are represented in FIG. 7C. Western blot analysis to determine VCAM1 protein expression was carried out (FIG. 7D). Similar experiments were carried out with HUVECs treated with mm-LDL and SOAE. Gene expression of VCAM1 (FIG. 8E) and MCP-1 (FIG. 7F) were analyzed. Results are representative of three experiments. Error bars represent SEM. *P<0.05; **P<0.01.

FIGS. 8A and 8B show SOAE inhibits NF-κB transcription and translocation. PCR array results showed that SOAE inhibited LPS-induced NF-κB mRNA levels in RAW 267.4 cells (FIG. 8A). Immunofluorescence staining revealed that SOAE inhibited NF-κB translocation to the nucleus in both macrophages (FIG. 8B-Upper row) and human umbilical vein endothelial cells (FIG. 8B-Lower row). Images are at 100× and are representative of three independent experiments.

FIGS. 9A through 9F show that SOAE inhibits oxidation of lipoproteins. LDL and HDL were isolated from human plasma and oxidation was carried out using copper (FIG. 9A and FIG. 9D); MPO (FIG. 9B and FIG. 9E) and MPO with tyrosine (FIG. 9C and FIG. 9F) in the presence or absence of SOAE. In all cases, SOAE effectively delayed or inhibited oxidation of lipoproteins in a concentration dependent manner.

FIGS. 10A through 10F show the differential regulation of expression of scavenger receptors and reverse cholesterol transport genes by SOAE. RAW cells were treated with increasing concentration of SOAE following which expression of SR-A1 and CD36 was analyzed. While SR-A1 mRNA expression was found to decrease with increasing concentration of SOAE (FIG. 10A), CD36 mRNA expression markedly increased (FIG. 10B). The protein expression of SRA1 and CD36 (FIG. 10C) corroborated with the real-time PCR results. The effect of SOAE on reverse cholesterol transport genes was analyzed by real-time PCR (FIG. 10D). Using HepG2-LXR reporter system the effect of SOAE on GFP expression was assayed; the left most panel are the bright field images; the middle panel represent fluorescence images and right most panel are the merged images (FIG. 10E). Luciferase activity (FIG. 10F) was also assayed in these cells. Results are average of three independent repeats. *P<0.05; **P<0.01.

FIGS. 11A and 11B are graphs showing TNF-α and IL-6 levels measured by ELISA from serum of mice as described herein. As shown in FIG. 11A, pretreatment of mice with 50 μg and 100 μg SOAE (**P<0.003) showed a significant reduction in LPS-induced TNFα levels as compared to LPS-treatment alone. A similar result was observed with IL-6 at 10 μg SOAE (**P<0.004). However, a dose-dependent increase in IL-6 (FIG. 11B) levels was observed at concentrations higher than 10 μg SOAE.

FIG. 12 is a table of primers used for real-time PCR analysis to measure mRNA levels of GAPDH, IL-1a, IL-6, TNF-a, MCP-1, and VCAM1.

### DETAILED DESCRIPTION

Disclosed herein are methods and compositions for extracting useful components associated with sesame seed oil (SO), including but not limited to a sesame seed oil aqueous extract (SOAE). Also provided are methods for treating, ameliorating, modulating or preventing complications and pathologies of conditions related to inflammation, including but not limited to atherosclerosis.

The materials, compounds, compositions, articles, and methods described herein can be understood more readily by reference to the following detailed description of specific aspects of the disclosed subject matter and the Examples and Figures included therein.

Before the present materials, compounds, compositions, articles, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings:

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an extract” includes mixtures of two or more such extracts, reference to “the oil” includes mixtures of two or more such oils, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the term “subject” refers to the target of administration, e.g., an animal. Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects. In some aspects of the disclosed methods, the subject has been diagnosed with a need for treatment for an inflammatory disorder, such as, for example, atherosclerosis.

As used herein, the term “treatment” refers to the medical management of a subject or a patient with the intent to cure, ameliorate, stabilize, or prevent a disease, pathological condition, or disorder, such as, for example, an injured myocardium. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In an aspect, the disease, pathological condition, or disorder is cardiac dysfunction, such as, for example, a cardiac ischemia/reperfusion event.

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by compositions or methods disclosed herein. For example, “diagnosed with an inflammatory disorder” means having been subjected to a physical examination by a person of skill, for example, a physician, and found to have a condition that can be diagnosed or treated by a compound or composition that alleviates or ameliorates inflammation.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to inflammation) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

The phrase “comestible composition” is meant a composition that is eaten, drank, or ingested by a human or other animal.

As used herein, the terms “administering” and “administration” refer to any method of providing the compositions disclosed herein to a subject. Such methods are well known to those skilled in the art and include, but are not limited to, intracardiac administration, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts.

The term “pharmaceutically acceptable” describes a material that is not biologically or otherwise undesirable, i.e., without causing an unacceptable level of undesirable biological effects or interacting in a deleterious manner. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of from about 0.01 to about 10 micrometers.

As used herein the term “sesame seed oil” is used to comprehensively encompass all kinds of sesame seed oil currently available. Accordingly, “sesame seed oil” includes but is not limited to, cold pressed sesame seed oil (extraction process involves application of low-temperature pressure to sesame seeds resulting in maximum preservation of nutrients), dark sesame seed oil (the oil is extracted from toasted sesame seeds, has a dark coffee-like hue), light sesame seed oil (extracted by applying pressures to raw sesame seeds until they produce oil. In certain aspects, such as in the experiments detailed in the Examples, cold pressed sesame seed oil is used.

Disclosed are the components to be used to prepare compositions of the subject matter disclosed herein as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

Reference will now be made in detail to specific aspects of the disclosed materials, compounds, compositions, articles, and methods, examples of which are illustrated in the accompanying Examples and Figures.

### Compositions and Methods

Inflammation-related conditions comprise conditions in the bodies of humans or animals that are due to, or related to, the presence of a complex biological response of vascular tissues to harmful stimuli such as pathogens, damaged cells, irritants or toxins and include, but are not limited to, inflammatory responses, atherosclerosis, Alzheimer's disease, allergies, myopathies, leukocyte defects, cancer, cardiovascular diseases, inflammatory bowel diseases, pelvic inflammatory diseases, and inflammatory diseases in the brain. Disclosed herein are compositions and methods that treat or prevent inflammation-related conditions. Atherosclerosis is a disease in which chronic inflammation is considered as an independent risk factor (Hansson, et al., The immune response in atherosclerosis: a double-edged sword. Nat Rev Immunol 2006; 6:508-519; Libby et al., Inflammation and Atherosclerosis. Circulation. 2002; 105:1135-1143; Packard et al., Inflammation in atherosclerosis: From vascular biology to biomarker discovery and risk prediction. 2008, Clinical Chemistry; 54:1, 24-38). A number of inflammatory markers such as cytokines (IL-1, IL-6, TNF, IL-12, IL-18), adhesion molecules, C-reactive protein (CRP), enzymes (myeloperoxidase-MPO, secretory phospholipase A2-sPLA2) have been found to be highly expressed in human and animal atherosclerotic lesions (Ridker et al., C-reactive protein and other markers of inflammation in the prediction of cardiovascular disease in women. N Engl J Med. 2000; 342:836-843; Danesh et al., C-reactive protein and other circulating markers of inflammation in the prediction of coronary heart disease. N Engl J Med. 2004; 350:1387-97; Vasan. Biomarkers of cardiovascular disease: molecular basis and practical considerations. Circulation. 2006; 113:2335-62; Biasucci et al., Inflammatory markers, cholesterol and statins: Pathophysiological role and clinical importance. Clin Chem Lab Med. 2010; 48(12):1685-1691). There is also considerable evidence for the involvement of immune and inflammatory response in atherosclerosis. It is well established that monocyte chemoattractant protein-1 (MCP1), a chemokine, is produced to recruit monocytes (Quinn et al., Oxidatively modified low density lipoproteins: a potential role in recruitment and retention of monocyte/macrophages during atherogenesis. Proc Natl Acad Sci USA. 1987; 84(9):2995-8; Cushing et al., Minimally modified low density lipoprotein induces monocyte chemotactic protein 1 in human endothelial cells and smooth muscle cells. Proc Natl Acad Sci USA. 1990; 87(13):5134-8) to the intima and adhesion molecules increase their attachment to activated endothelial cells (Li et al., An atherogenic diet rapidly induces VCAM-1, a cytokine regulatable mononuclear leukocyte adhesion molecule, in rabbit endothelium. Arterioscler Thromb. 1993; 13:197-204; Galkina et al., Vascular adhesion molecules in atherosclerosis. Arterioscler Thromb Vasc Biol. 2007; 27:2292-2301). Monocyte-macrophage heterogeneity (Gordon et al., Monocyte and macrophage heterogeneity. Nat Rev Immunol. 2005; 5(12):953-64; Selvarajan et al., Peritoneal macrophages are distinct from monocytes and adherent macrophages. Atherosclerosis. 2011; 219(2):475-83) adds to the complexity of the process wherein pro-inflammatory monocytes augment the immune response through the expression of pro-inflammatory cytokines and other factors such as proteases (Swirski et al., Ly-6Chi monocytes dominate hypercholesterolemia-associated monocytosis and give rise to macrophages in atheromata. J Clin Invest 2007; 117:195-205; Khallou-Laschet et al., Macrophage plasticity in experimental atherosclerosis. PLoS One. 2010 5(1): e8852; Wilson. Macrophages heterogeneity in atherosclerosis-implications for therapy. J Cell Mol Med. 2010; 14(8):2055-65). Thus anti-inflammatory therapy as a means to control atherosclerosis has long been in consideration.

Sesame seed oil (SO) obtained from Sesamum indicum is rich in both monounsaturated and polyunsaturated fatty acids. It contains approximately 47% oleic acid and 39% linoleic acid (Grishina et al., Fatty acid composition of oils from different varieties of sunflower, peanut and sesame. Vopr Pita 1970; 29:81-88; Sengupta et al., Triglyceride composition of Sesamum indicum seed oil. J Sci Food Agric 1976; 27:165-169). It also contains lignans such as sesamin and sesamolin and several antioxidant compounds such as sesamol and sesaminol (Egbekun et al., Proximate composition and functional properties of full fat and defatted beniseed (Sesamum indicum L.) flour. Plant Foods Hum Nutr 1997; 51:35-41) and other methylenedioxyphenol derivatives. SO has been reported to help reduce high blood pressure and lower the amount of medication needed to control hypertension (Sankar et al., Modulation of blood pressure, lipid profiles and redox status in hypertensive patients taking different edible oils. Clin Chim Acta 2005; 355:97-104; Sankar et al., Effect of sesame seed oil on diuretics or Beta-blockers in the modulation of blood pressure, anthropometry, lipid profile, and redox status. Yale J Biol Med. 2006; 79(1):19-26.). Other beneficial effects include reduction in plasma cholesterol, LDL cholesterol and triglyceride (TG) levels (Satchithanandam et al., Effect of sesame seed oil on serum and liver lipid profiles in the rat. Int J Vitam Nutr Res. 1996; 66:386-392). Earlier studies by the inventors in SO-diet fed LDLR−/− mice showed that the plasma levels of total cholesterol, triglycerides, VLDL cholesterol, and LDL cholesterol were decreased while HDL was significantly increased in these animals as compared to atherosclerotic diet-fed animals. The SO-containing diet effectively prevented atherosclerosis lesion formation in LDLR−/− male mice (Bhaskaran S, Santanam N, Penumetcha M, Parthasarathy S. Inhibition of atherosclerosis in low-density lipoprotein receptor-negative mice by sesame seed oil. J Med Food. 2006; 9(4):487-90). The fatty acid composition of SO is roughly in between those of olive and sunflower oils and yet the observed level of inhibition of atherosclerosis was significantly higher. This prompted further discovery of components in addition to fatty acid unsaturation responsible for the observed level of inhibition. As disclosed in greater detail in the Examples, an aqueous extract of SO was identified and tested to determine if it would be effective in inhibiting inflammation. In addition, the effect of SOAE on regulating lipid metabolism was also investigated by studying expression of reverse cholesterol transport genes (RCT) and on macrophage scavenger receptors as a complex interplay of all these factors are involved in atherosclerosis.

Even though sesame seed oil is an edible oil containing triglycerides and other lipid components, many non-oil components with varying levels of solubility in aqueous solvents are associated with SO. Disclosed herein is a unique methodology for separating the non-lipid components of sesame seed oil. Sesame seed oil aqueous extract (SOAE) was prepared by using sesame seed oil and distilled water. In summary, a combination of sesame seed oil and distilled water was mixed vigorously at room temperature in the dark. The aqueous portion was separated by repeated filtration using filter paper such as Whatman filter paper and lyophilized. The lyophilized sample was reconstituted with pyrogen-free water and absence of possible endotoxin contamination of SOAE was confirmed using limulus assay. The SOAE was characterized by UV spectral analysis along with sesamol and sesamin. After confirming the absence of considerable lipid content (by TLC) or protein content (by SDS PAGE gel), the aqueous extract was used for in vitro studies.

As detailed in the Examples, sesame seed oil aqueous extracts were tested to determine if they was anti-inflammatory and whether it would inhibit bacterial lipopolysaccharide (LPS)-induced pro-inflammatory cytokine production by macrophages. The results showed that SOAE has potent anti-inflammatory properties as seen with LPS-induced inflammation in RAW macrophages or mouse peritoneal macrophages. The atherosclerosis PCR gene array revealed down-regulation of a number of inflammatory markers such as Ccl2 or MCP-1, Ccl5 or RANTES, IL-1α, IL-1β and TNF in the presence of SOAE. Other targets that are thought to play a role in the atherosclerotic disease process such as colony stimulating factor 2 (Csf2), Nfkb1, an important pro-inflammatory transcription factor, and matrix metalloproteinase 3 (Mmp3) were also suppressed. Independent analysis also revealed significant inhibition of IL-6 expression in the presence of SOAE. Therefore, though not wishing to be bound by the following theory, it is believed that components in SOAE regulate many inflammatory pathways. This anti-inflammatory property is unique to SOAE as aqueous extract of other oils, such as sunflower oil, did not show any effect on LPS-induced inflammatory markers in macrophages. Though not wishing to be bound by the following theory, it is believed that SOAE has components that inhibit inflammation through blocking NF-κB activation. SOAE also effectively inhibited oxidation of LDL and HDL by Cu and myeloperoxidase (MPO). In more recent studies, SOAE significantly prevented atherosclerosis in a mouse model.

Dietary intervention to delay and prevent chronic inflammatory diseases is more desirable than pharmacological intervention. Such approaches could lead to the development of inexpensive and powerful “adjunct therapy” to existing medications. It has been surprisingly found that SOAE as described herein demonstrates anti-inflammatory properties comparable to, or better than, sesame seed oil by itself or other oil extracts. The SOAE compositions disclosed herein can comprise dry powders, liquid formulations, food or beverage compositions, cosmetic compositions, compositions administered by injection, compositions administered transdermally, and can further be incorporated into articles such as toothpaste and wound healing devices (bandaids, bandages etc.). The compositions can be added to a second composition to form a combined composition to provide enhanced anti-inflammatory activity.

Liquid anti-inflammatory compositions disclosed herein comprise a SOAE extract composition, in either a dry powder form or a liquid form, and a liquid, including but not limited to, water, which can be still or carbonated, and other ready to drink or ready to mix beverages, including but not limited to coffees, teas, energy drinks, juices, milks, and plant liquids such as soy products, sugar cane products, coconut products, protein drinks, meal replacement drinks, and alcohol containing products such as beer and wine. Liquid anti-inflammatory compositions can comprise pharmaceutical, nutraceutical or dietary supplement compositions in combination with a SOAE extract composition. For example, an anti-inflammatory composition can comprise liquid pharmaceutically acceptable syrups, excipients, fillers or other known pharmaceutical formulations in combination with a SOAE extract composition. As a further example, an anti-inflammatory composition can comprise a SOAE extract composition, in a pharmaceutical formulation for ocular drops to provide anti-inflammatory effects to the eye, for example, for treating seasonal allergies or for treating vision impairment.

Solid or dry anti-inflammatory compositions can comprise a SOAE extract composition, and a solid or dry material, such as a food product for ingestion by a human or animal. Solids or dry materials include, but are not limited to, foods, nutritive and non-nutritive sweeteners, pharmaceutical, nutraceutical or dietary supplement formulations. For example, an anti-inflammatory composition can comprise solid or dry pharmaceutically acceptable compositions, excipients, fillers or other known pharmaceutical formulations, to be made into dosage units such as tablets, capsules or powders, in combination with a SOAE extract composition. The compositions disclosed herein can function as additives to foods, and be combined with food products, including foods wherein a dry or liquid a SOAE extract composition can be added, so as to provide anti-inflammatory activity to the food.

Some specific examples of foods that can be combined with SOAE disclosed herein include, chocolate confectionery, boiled sweets, gums, jellies and chews, lollipops, gum, bread, pastries, cakes, biscuits, crackers, cereals, fruit snacks, chips, pretzels, dessert mixes, soup (e.g., canned soup, dehydrated soup, instant soup, chilled soup, frozen soup), pasta, noodles, canned food, sauces, dressings, spreads, jams and preserves.

Some other specific examples of suitable beverage include alcoholic and non-alcoholic beverages, coffee, tea, fruit juice, vegetable juice, milk, energy drink, protein drink, soft drinks, and alcoholic drinks.

A SOAE powder can be added in compositions disclosed herein in an amount from about 0.001 mg to about 1000 mg, w/w, or to liquid compositions in an amount from about 0.001 mg/mL to about 1000 mg/mL. A SOAE can be added to a composition in a liquid form (for example, prior to lyophilization) or can be added to a composition in its powdered form (post-lyophilization).

As a specific example, an anti-inflammatory composition can be added to a food product for human or animal consumption. An anti-inflammatory sweetener can comprise a SOAE extract composition, or in combination with a compound or composition that has a sweet taste, or is a recognized sweetening agent. Anti-inflammatory compositions can be used as a sweetening agent, and can comprise a SOAE extract composition combined with sweeteners, including but not limited to sweet natural compounds or compositions such sucrose or maple syrup, or artificial sweeteners. The anti-inflammatory SOAE extract composition can be combined with a sweetener, to form a SOAE extract composition, an anti-inflammatory sweetener. A SOAE extract composition can be added to a sweetener compound or composition, and used for example, as a coating in a continuous spray agglomeration to sugar granules, or added to an alternative natural sweetener such as erythritol, to provide the sweetener with anti-inflammatory properties. The SOAE extract composition can be added as a liquid to a stevia extract to provide a combined stevia/mineral extract composition sweetener with anti-inflammatory properties.

A SOAE anti-inflammatory composition can be provided or administered to a subject once a day, two times a day, three times a day or more often. An anti-inflammatory composition can be administered daily, weekly, or monthly in a regular schedule or on an as needed schedule. In general, an anti-inflammatory composition comprising a SOAE can comprise from about 0.0001 g to about 1000 g of the SOAE, or from about 0.0001 g to about 100 g, from about 0.0001 g to about 10 g, from about 0.001 g to about 1000 g, from about 0.01 g to about 1000 g, from about 0.1 g to about 1000 g, from about 1.0 g to about 100 g, from about 1.0 g to about 10 g, from about 10 g to about 1000 g, and ranges therein between. For example, a 12 oz enhanced water beverage can contain about 0.1 gram of a SOAE extract composition, while a 10 oz carbonated beverage can contain about 10 grams of a SOAE extract composition, and a gallon can contain about 300 grams of a SOAE extract composition.

Disclosed herein are SOAE compositions used in methods for treating, ameliorating, modulating or preventing inflammation, methods of inhibiting formation of inflammation end products, inhibition of inflammation reactions of proteins, lipids and/or nucleic acids, inhibition of painful effects related to inflammatory reaction, and methods for treatment or prevention of inflammation-related conditions including, but not limited to, complications of atherosclerosis, rheumatoid arthritis, Alzheimer's disease, diabetes (Type I and II), uremia, neurotoxicity, inflammatory reactions, ventricular hypertrophy, angiopathy, myocarditis, nephritis, arthritis, glomerulonephritis, microangiopathies, and renal insufficiency.

Methods disclosed herein comprise making and using the disclosed anti-inflammatory compositions. Such compositions can be consumed by healthy young and adult animals and humans, as well as humans or animals at risk for developing, or suffering from, atherosclerosis or similar inflammatory-related conditions. Food, beverages, and nutritional supplement anti-inflammatory compositions can be used to provide anti-inflammation potential to a subject and provide a benefit in promoting health and wellness. For example, a line of food or beverages having anti-inflammatory capability comprising a SOAE extract composition disclosed herein can be used by different ages for multiple health benefits.

Disclosed herein are methods for amelioration, modulation, treatment or prevention of inflammation-related conditions, comprising providing a SOAE composition that can be ingested by subjects, animals or humans, to reduce the effects of inflammation on the linings of the gastrointestinal system. Such anti-inflammatory compositions can support intestinal anti-inflammatory effects, enhance intestinal detoxification of oxidative compounds, promote intestinal nutrient absorption, help retard intestinal inflammatory processes, encourage intestinal health and help reduce the factors of inflammation, and aid in calming and soothing intestinal inflammation. Beverages or foods, in addition to comprising an anti-anti-inflammatory composition, can also comprise botanicals such as chamomile, dandelion, echinecea, milk thistle, gentian, licorice, chickenweed, meadowsweet, goldenseal, spanish black radish, and chlorophyll.

Methods for amelioration, modulation, treatment or prevention of inflammation-related conditions, or inhibition of inflammatory products include providing an effective amount of a SOAE composition for arthritis. Advanced inflammatory products are thought to be promoters of inflammation and eventual joint degradation as seen in osteoarthritis and rheumatoid arthritis. Activated inflammation mediators called MMPs begin the cascading effect of pain and limited movement flexibility. Synovial fluids become oxidized and subject to hardening. Markers of inflammation have been identified as present in elevated amounts in synovial fluids of osteoarthritis and rheumatoid arthritis patients indicating inflammation is a factor in these conditions. Prophylactic use of anti-inflammatory compositions disclosed herein at an early age can prevent or delay the onset of these conditions and, intake at any age can reduce the incidence and severity of osteoarthritis and rheumatoid arthritis. Taken at any age, the anti-inflammatory compositions comprising SOAE can limit the cascading damage of inflammation and thus reduce the propensity to promote longer term arthritic diseases that come with aging. For example, a beverage or a food comprising an anti-inflammatory composition comprising a sesame seed oil aqueous extract can be ingested by subjects, animals or humans, to reduce the effects of inflammation on the joints or synovial fluid. Such compositions can be used to retard age-related arthritic degeneration, maintain joint and tendon flexibility, promote healthy bone strength and joint elasticity, encourage bone structure integrity and flexibility, defend against inflammation-induced joint degradation, promote healthy synovial environment to joints and tendons, encourage more active lifestyle when living with arthritis, promote longer preventive wellness for arthritis, and reduce incidence of inflammation, point swelling and tightness.

Methods of treating, ameliorating, modulating or preventing of inflammation-related conditions, include providing an effective amount of an anti-inflammatory composition comprising a SOAE extract for improving cognitive function. Cognition is a general term covering many aspects of brain function, including learning, remembering, thinking and reasoning. These processes can decline during the natural aging process or in the event of degenerative disease, such as brain inflammatory disease. For example, a beverage or food comprising an anti-inflammatory composition comprising a sesame seed oil aqueous extract can be ingested by subjects, animals or humans, to reduce the effects of inflammation on the nervous system, reduce inflammatory reactions, and prevent damage to the circulatory system. Such anti-inflammatory compositions comprising a sesame seed oil aqueous extract can be used to protect against cognitive degradation, restore optimal cognitive functionality, encourage healthy cognitive function, enhance cognitive performance, promote healthy brain function, aid in combating oxidative-induced cognitive degradation, strengthen cognitive function defense, and to stimulate coherent cognitive processes. Beverages or foods, in addition to comprising an anti-inflammatory composition comprising a sesame seed oil aqueous extract, can also comprise herbal or botanical compounds or extracts of ginko biloba, ginseng, vipocetine, green tea, soy isoflavones, Vitamins E, C, B6, B12, phospholipids (phosphatidylserine and phosphatidylcholine) and citocoline (a precursor) and glycerophosphocholine, alpha lipoic acid, acetyl-L-carnitine, coenzyme Q10, creatine, essential fatty acids, DHA, EPA, and resveratrol, or grapeseed extract.

Beverages or foods, in addition to comprising an anti-inflammatory composition comprising a sesame seed oil aqueous extract, can also comprise herbals or extracts of herbals such as ginger, chinese thunder god vine, willow bark extract, feverfew, cat's claw, stinging nettle, boswellia, turmeric, S-adenosylmethionine (SAMe), chondroitin sulfate, glucosamine, essential fatty acids, and enzymes, such as bromelain, and quercetin.

Beverages or foods, in addition to comprising an anti-inflammatory composition comprising a sesame seed oil aqueous extract, can also comprise Vitamin C and E, bioflavonoids, essential fatty acids, yohimbe bark, horny goat weed, maca, saw palmetto, and man bao.

Anti-inflammatory compositions comprising a sesame seed oil aqueous extract for these and other inflammation-related conditions can comprise ready-to-eat-cereals, fruit juices, candy bars, chewing gum, nutritional supplements, enhanced water beverages, carbonated and non-carbonated drinks, alcoholic beverages such as beer and wine, baby food, and many other foods and beverages. For example, the SOAE can be chilled and added to carbonated drinks. The SOAE can also be frozen and used as “ice-cubes” for beverages. The anti-inflammatory compositions comprising a SOAE disclosed herein can also be used an animal feed additive.

Disclosed herein is a method for making a sesame seed oil aqueous extract, comprising admixing sesame seed oil with water in a 1:0.25 ratio, shaking the mixture, filtering the mixture to separate at least a portion of the lipids to form a filtrate substantially free of lipids.

Disclosed herein is a method for making a sesame seed oil aqueous extract, comprising admixing sesame seed oil with water in a 1:0.25, shaking the mixture, filtering the mixture to separate at least a portion of the lipids to form a filtrate substantially free of lipids, further comprising, dehydrating the filtrate substantially free of lipids to form an aqueous exact of sesame seed oil powder.

Disclosed herein is a method for making a sesame seed oil aqueous extract, comprising admixing sesame seed oil with water in a 1:0.25 ratio, shaking the mixture, filtering the mixture to separate at least a portion of the lipids to form a filtrate substantially free of lipids, wherein filtering comprises allowing the mixture to flow through a filter paper of 2-12, 3-9, or 5 micron pore size.

Disclosed herein is a method for making a sesame seed oil aqueous extract, comprising admixing sesame seed oil with water in a 1:0.25 ratio, shaking the mixture, filtering the mixture to separate at least a portion of the lipids to form a filtrate substantially free of lipids, wherein the step of filtering is repeated more than one time, with the filtrate of the previous filtering step flowing through a new filter paper of 2-12, 3-9, or 5 micron pore size.

Disclosed herein is a method for making a sesame seed oil aqueous extract, comprising admixing sesame seed oil with water in a 1:0.25 ratio, shaking the mixture, filtering the mixture to separate at least a portion of the lipids to form a filtrate substantially free of lipids, wherein the shaking is performed in an opaque container.

Disclosed herein is a method for making a sesame seed oil aqueous extract, comprising admixing sesame seed oil with water in a 1:0.25 ratio, shaking the mixture, filtering the mixture to separate at least a portion of the lipids to form a filtrate substantially free of lipids, dehydrating the filtrate substantially free of lipids to form an aqueous exact of sesame seed oil powder. Disclosed herein are items of consumptions such as beverages, and foods comprising aqueous exact of sesame seed oil powder.

Disclosed herein are methods of treating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal.

Disclosed herein are methods of treating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal and wherein the composition is provided to the human or animal as a component of a beverage, or other consumable composition.

Disclosed herein are methods of treating or preventing inflammation-related conditions, comprising, administering to a human or animal an effective amount of an anti-inflammatory composition comprising a sesame seed oil aqueous extract composition, wherein the amount is effective in reducing at least a portion of the inflammatory events in a human or animal, wherein the composition is provided to the human or animal as a component of a beverage, or other consumable composition and wherein the inflammation-related condition is atherosclerosis.

Disclosed herein are methods of treating or preventing inflammation-related conditions, comprising, administering to a human or animal an effective amount of an anti-inflammatory composition comprising a sesame seed oil aqueous extract composition, wherein the inflammation-related condition comprises atherosclerosis, Alzheimer's disease, allergies, myopathies, leukocyte defects, cancer, cardiovascular diseases, inflammatory bowel diseases, pelvic inflammatory diseases, and inflammatory diseases in the brain.

Disclosed herein are methods and compositions for improved intravenous formulations comprising suitable nutritional supplements, including but not limited to, saline and/or glucose, wherein such formulations would have rapid action and be useful for treating conditions marked by inflammation, including but not limited to sepsis.

Disclosed herein are methods of treating or preventing inflammation-related conditions, comprising, proactively administering to a human or animal an effective amount of an anti-inflammatory composition comprising a sesame seed oil aqueous extract composition, wherein the composition is administered in a preventative capacity to delay, mitigate or prevent future development of chronic inflammatory diseases and conditions.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1

### Preparation of a Sesame Seed Oil Aqueous Extract (SOAE)

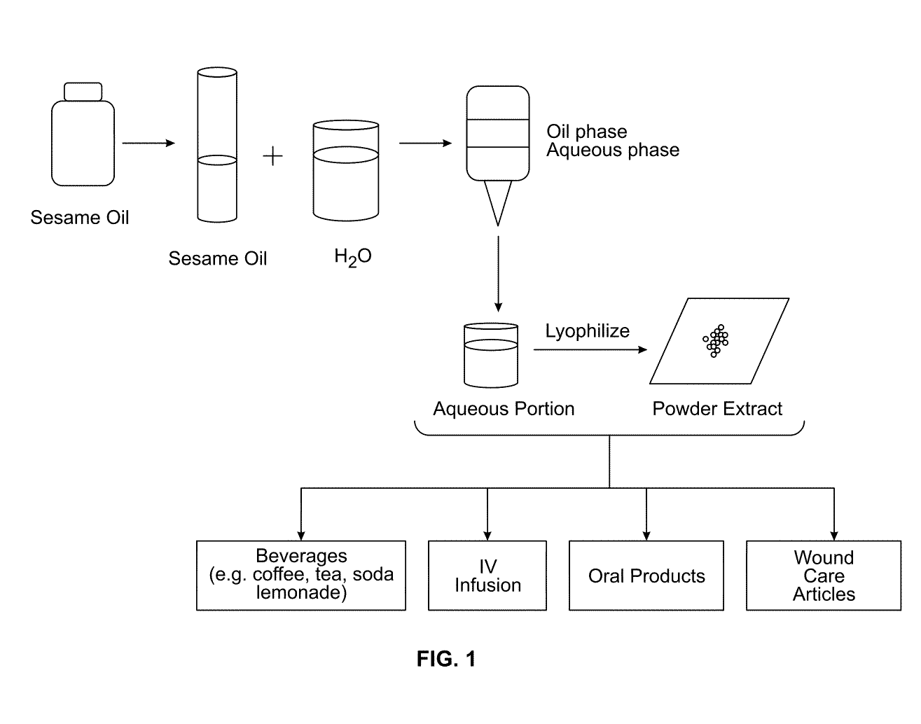

A sesame seed oil aqueous extract (SOAE) was prepared by mixing 1250 mL of sesame seed oil obtained from Sesamum indicum and 750 mL of nitrogen bubbled warm water (FIG. 1). The mixture was mixed vigorously in the dark at room temperature. The aqueous portion was separated from the oil portion by filtration (FIG. 1). The aqueous portion was then lyophilized (FIG. 1), and the lyophilized sample was reconstituted with pyrogen-free water to obtain a sesame seed oil aqueous extract. Absence of possible endotoxin contamination of SOAE was confirmed using a limulus assay.

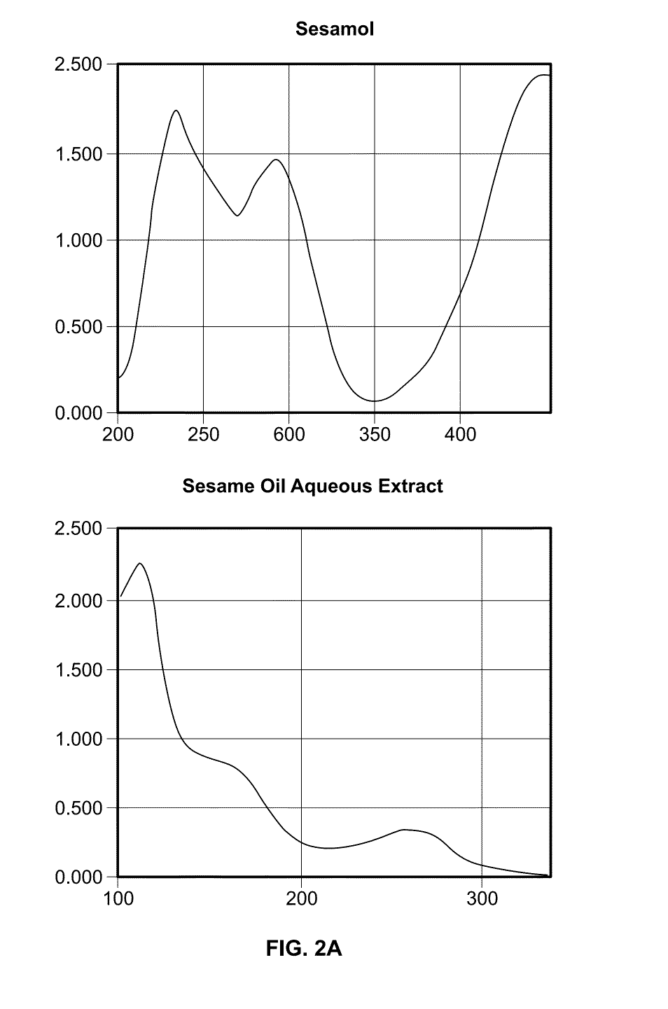

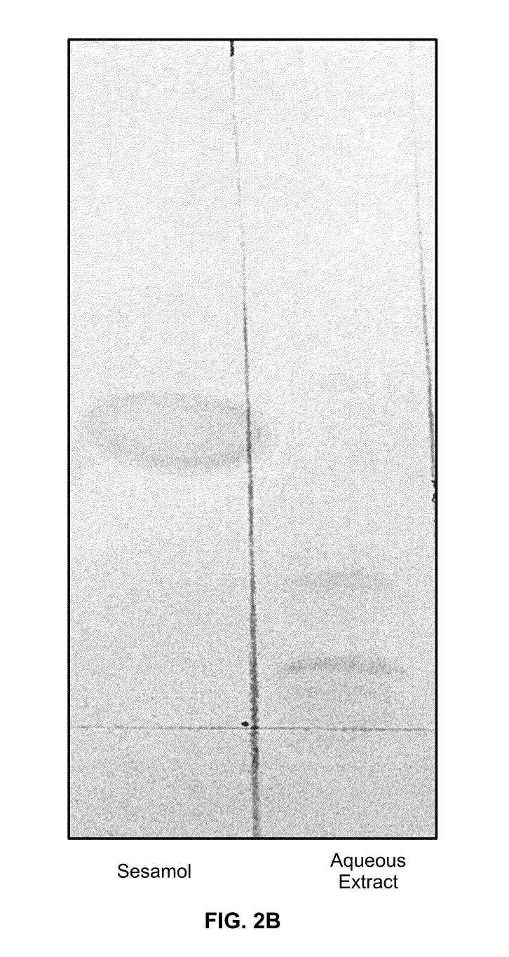

The SOAE was characterized by UV spectral analysis. An absorption spectral analysis using 10 μL of the aqueous extract was performed between 200 nm and 300 nm. An absorption spectral analysis of sesamol was also performed between 200 nm and 300 nm. FIG. 2A shows results of the absorption spectral analysis of sesamol and SOAE. The SOAE was further characterized by a thin layer chromatography (TLC) analysis. FIG. 2B shows results of the TLC analysis performed for sesamol and the SOAE. The results of the analyses indicated the SOAE contained additional components that are distinct from sesamol.

### Example 2

### Inhibition of LPS-Induced Inflammatory Cytokines in RAW Cells by Sesame Seed Oil Aqueous Extract (SOAE)

To study anti-inflammatory effects of SOAE, RAW 264.7 cells (macrophage cell line) were treated with the SOAE in the presence or absence of lipopolysaccharide (LPS) for 24 hours. Specifically, RAW 264.7 cells (ATCC macrophage cell line) were cultured in RPMI 1640 supplemented with 10% fetal bovine serum (FBS). Cells were plated in 6-well plates at a density of 1×10⁶ cells per well. Cells were pre-incubated in serum-free RPMI. They were then treated with the SOAE in the presence or absence of LPS for 24 hours.

RNA was extracted using TRIZOL™ reagent. mRNA expression of inflammatory cytokines such as IL-1α, IL-6 and TNF-α were analyzed by real time PCR.

Protein expression was determined by western blot analysis. Specifically, protein was isolated from the RAW cells using standard techniques. 25 μg of sample was fractionated on a 12% SDS polyacrylamide gel and transferred to a PVDF membrane for immunoblotting. Membranes were blocked with 5% milk in TBST followed by incubation with primary antibody overnight at 4° C. Anti-mouse antibodies were used for IL-6, IL-1α and β-actin. HRP-conjugated secondary antibodies were used. Protein bands were visualized using ECL according to manufacturer's instructions.

To identify the mechanism of action, a luciferase assay using HepG2-LXR reporter cell lines was performed. HepG2 reporter cell lines from System Biosciences are puromycin resistant stable reporter cell lines. They have a dual reporter vector system to quantitate Firefly Luciferase and GFP for live cell imaging. HepG2-LXR reporter cells were cultured in advanced DMEM with 10% FBS under selection conditions of 1 μg/mL puromycin. For the assay, cells were plated in 48-well plates at a density of 40,000 cells per well. They were then treated with LXR agonist T0901317 (5 nM, 50 nM, 500 nM and 1 μM) or SOAE (2.5 μL and 7.5 μL per mL). Cells were incubated overnight and the following day there were observed for GFP expression. Cells were then lysed and assayed for luciferase activity using the luciferase assay system from Promega (Madison, Wis.).

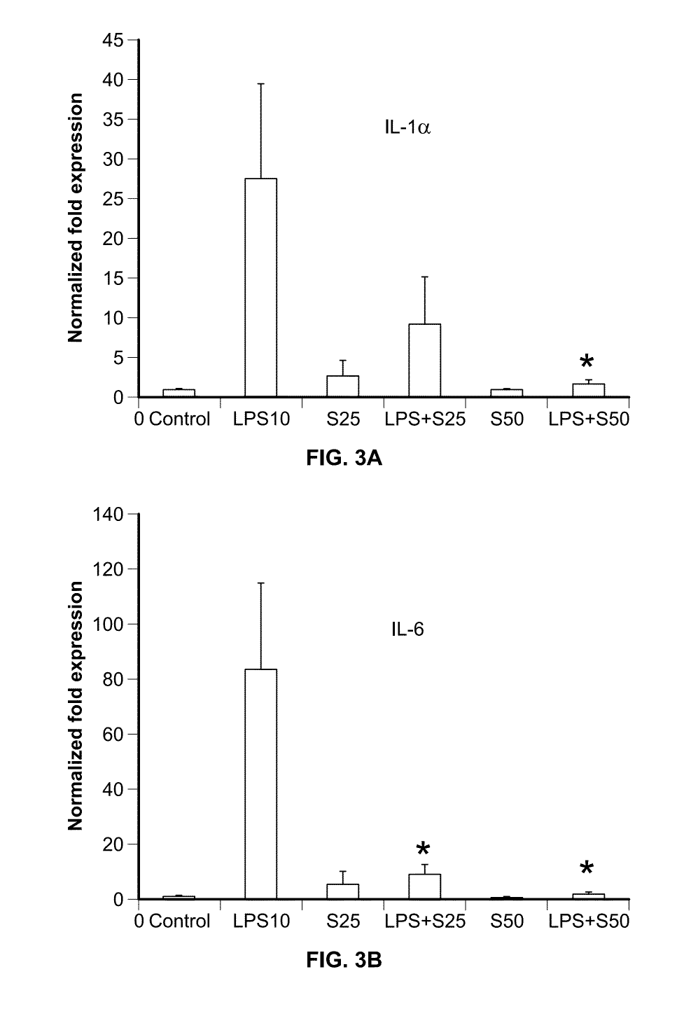

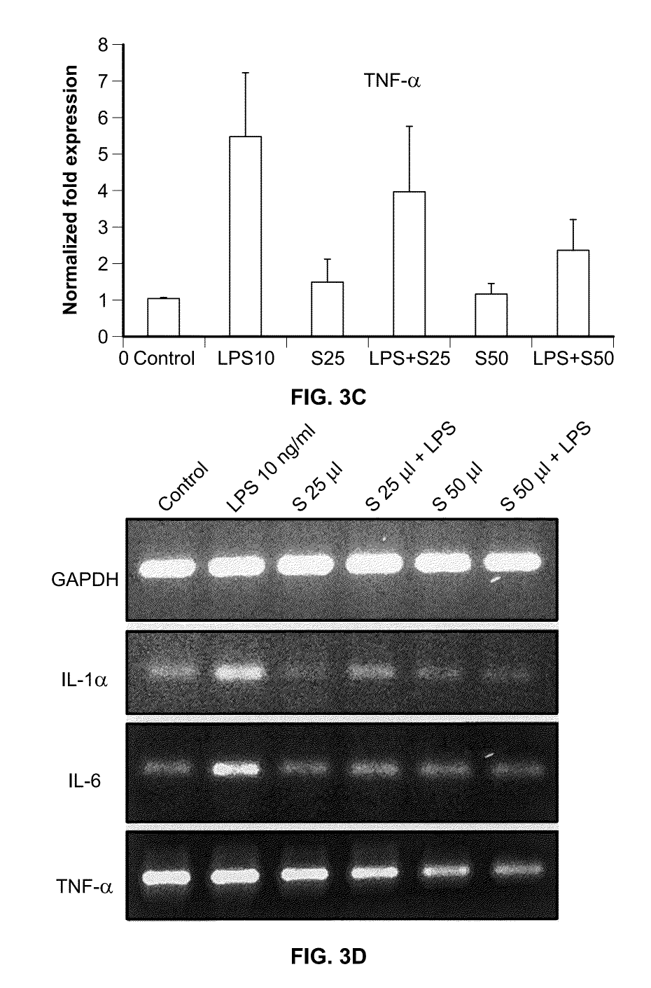

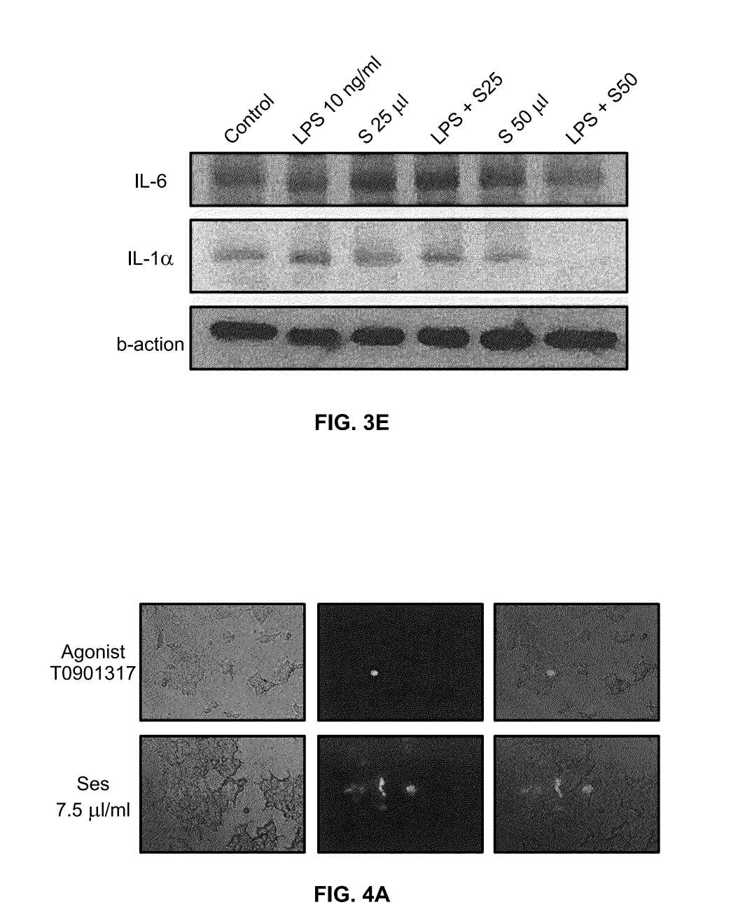

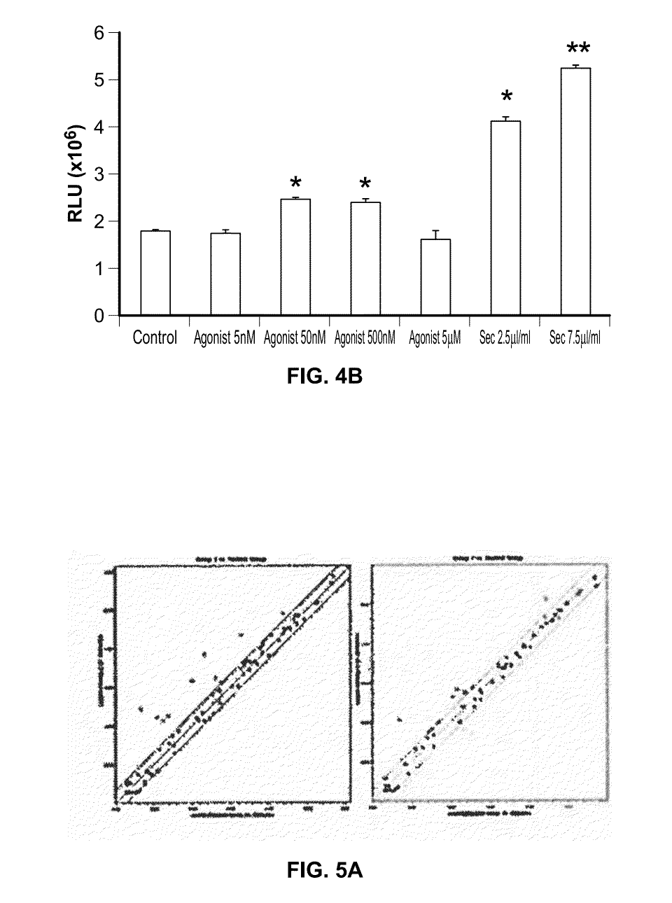

It was found that LPS induced the expression of IL-1α, IL-6 and TNF-α mRNA levels in RAW cells (FIGS. 3A through 3D). The SOAE alone did not significantly affect the expressions of inflammatory cytokine genes (FIGS. 3A through 3D). However, when treated together with LPS, SOAE inhibited the mRNA levels of these cytokines significantly (FIGS. 3A through 3D). Treatment with LPS together with SOAE also decreased the protein expression of these cytokines (FIGS. 3A through 3D). The SOAE induced LXR expression as identified by the luciferase assay system in HepG2-LXR reporter cells (FIGS. 4A and 4B).

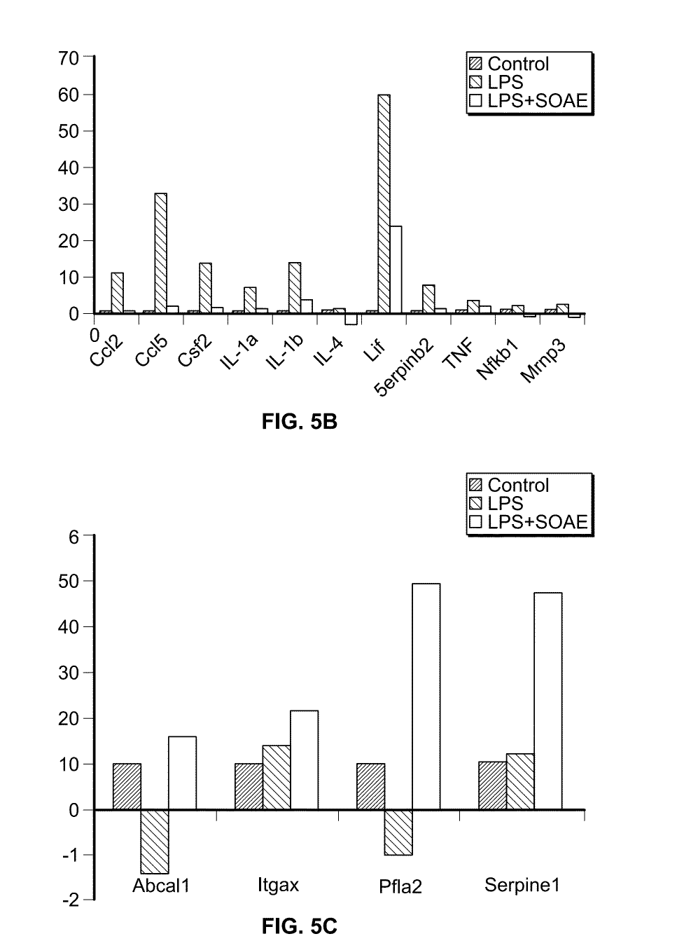

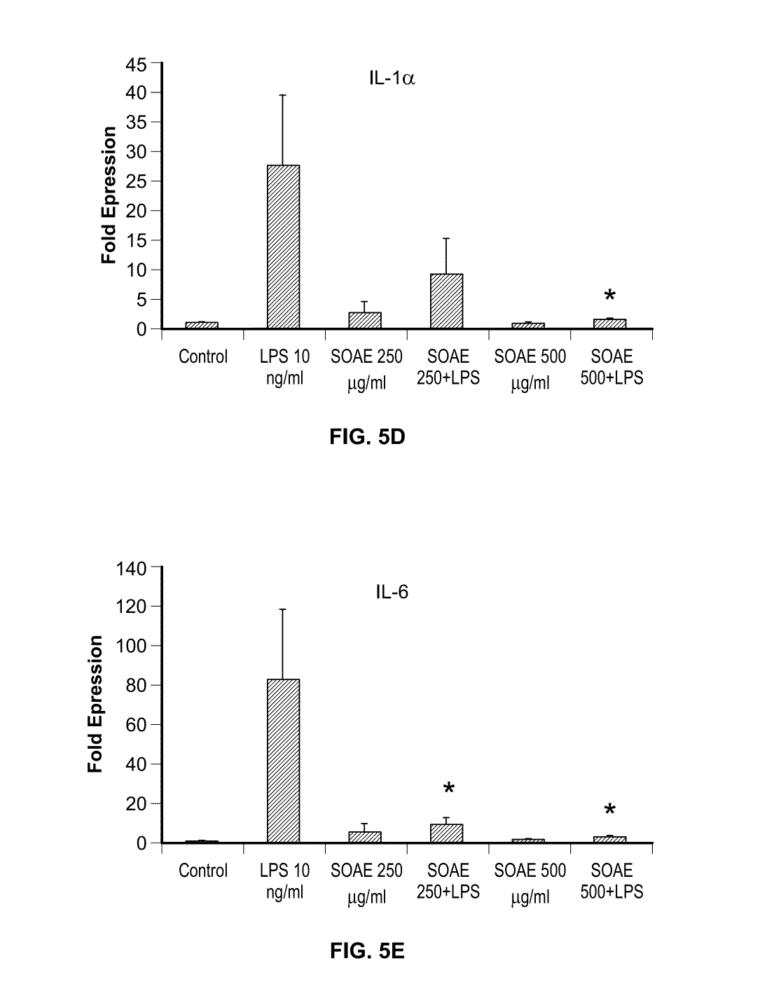

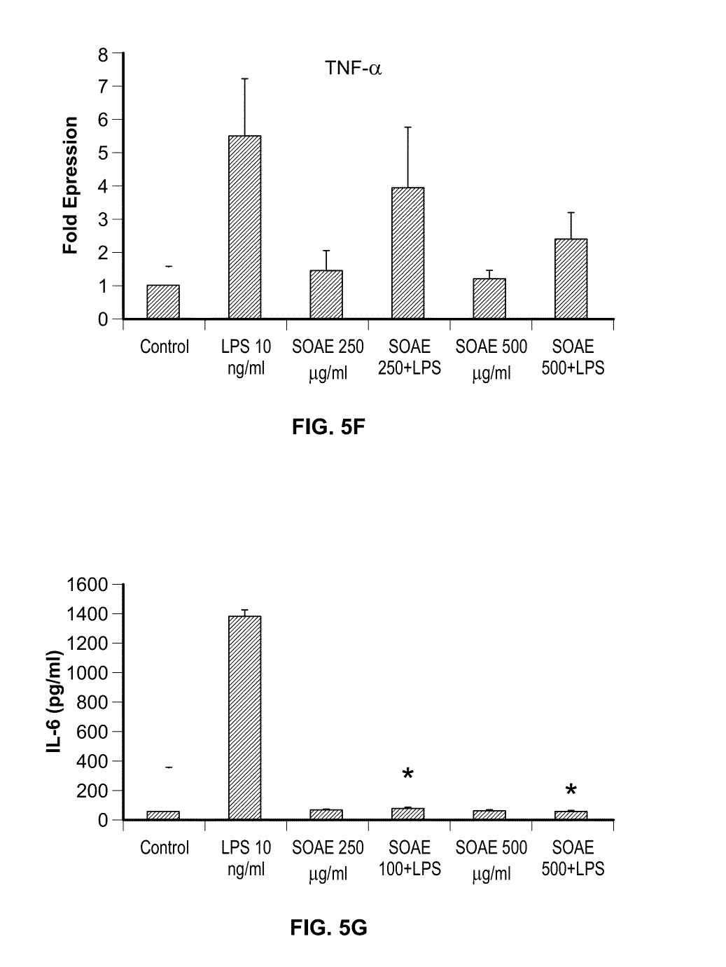

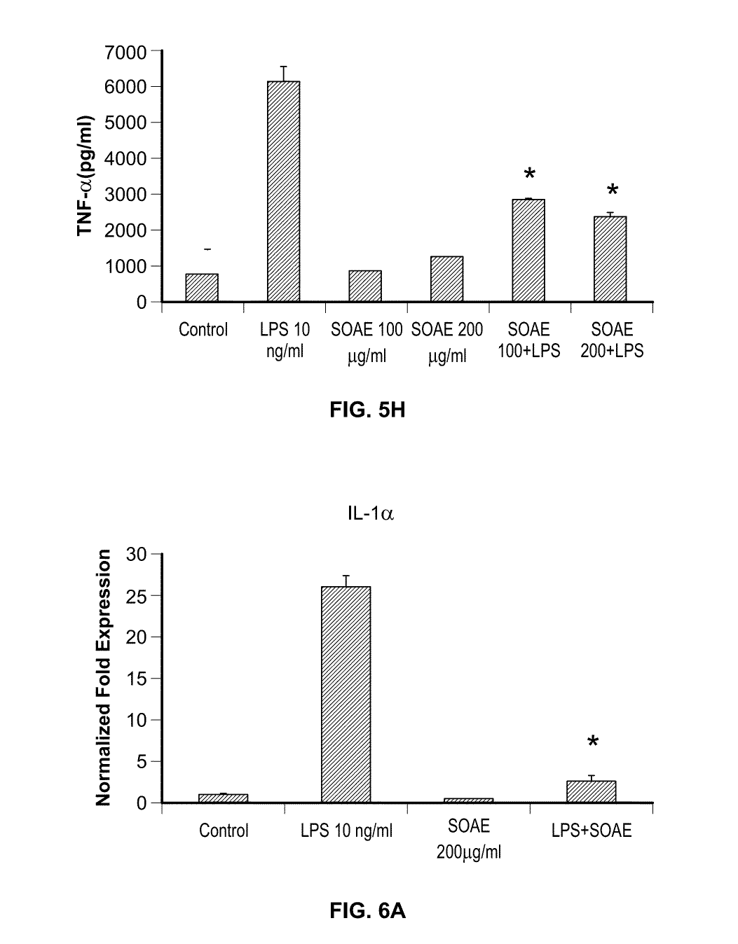

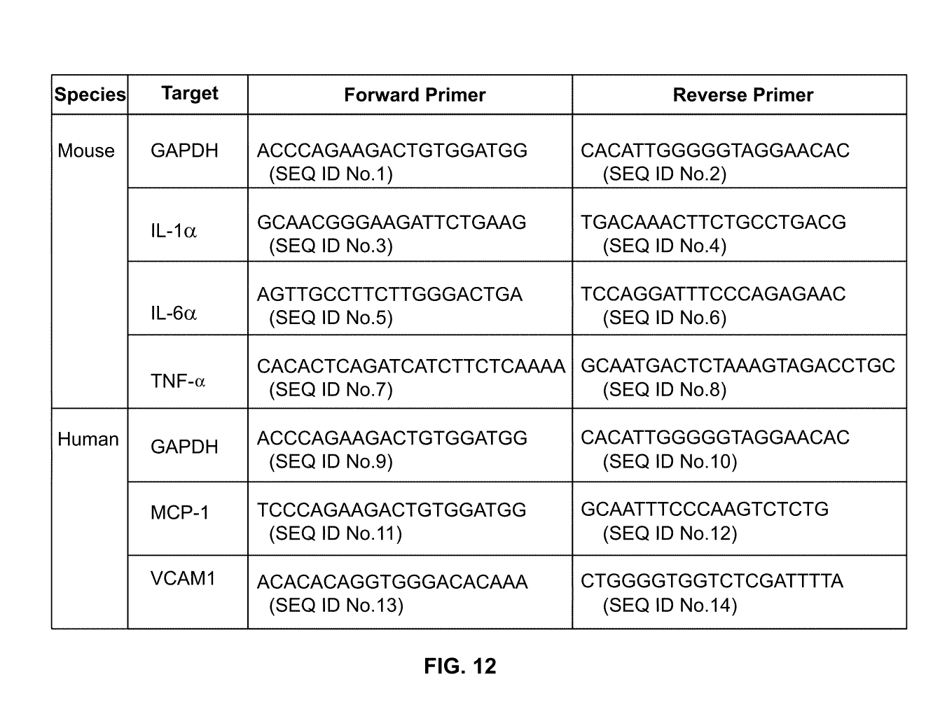

In another experiment to study the anti-inflammatory effects of SOAE, RAW cells were treated with LPS (10 ng/mL)±SOAE (250 and 500 μg/mL) for 24 hours. One μg of RNA was reverse transcribed into cDNA and quantitative real time PCR was performed with SYBR Green (Invitrogen, Carlsbad, Calif.). mRNA expression of inflammatory cytokines such as IL-1α, IL-6 and TNF-α was analyzed in macrophages. VCAM1 and MCP-1 expression was analyzed in HUVECs, which were cultured in M199 with 20% FBS. The primers for the genes are provided in FIG. 12. LPS strongly induced mRNA levels of IL-1α, IL-6, and TNF-α in RAW cells (FIGS. 5A through 5H). However in the presence of SOAE, the expression of these inflammatory markers was significantly inhibited in a concentration dependent manner. The extract alone did not induce any of the inflammatory cytokines.

The medium from cells treated with LPS (10 ng/mL)±SOAE (100 and 200 μg/mL) were assayed for IL-6 and TNF-α levels. 50 μL of samples were analyzed using sandwich ELISA kit (R and D, Minneapolis, Minn.) following suppliers protocol. Concentration of IL-6 and TNF-α was expressed in pg/mL. ELISA for IL-6 and TNF-α also supported mRNA expression results (FIGS. 5A through 5H). IL-6 and TNF-α secretion into the medium was enhanced when cells were treated with LPS alone. However presence of SOAE significantly inhibited LPS-induced secretion of the cytokines from RAW cells in a concentration dependent manner.

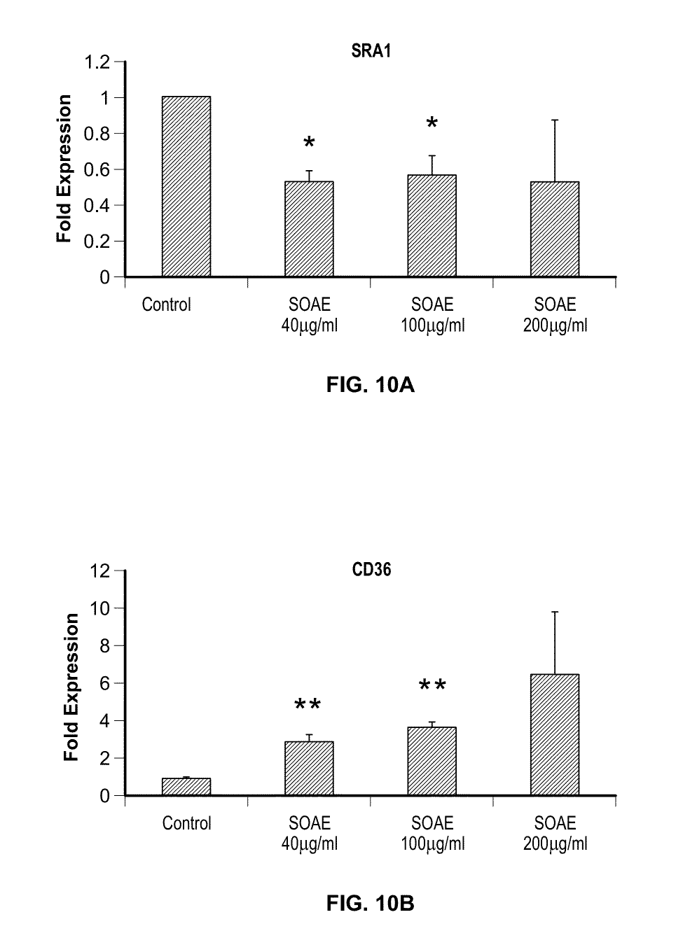

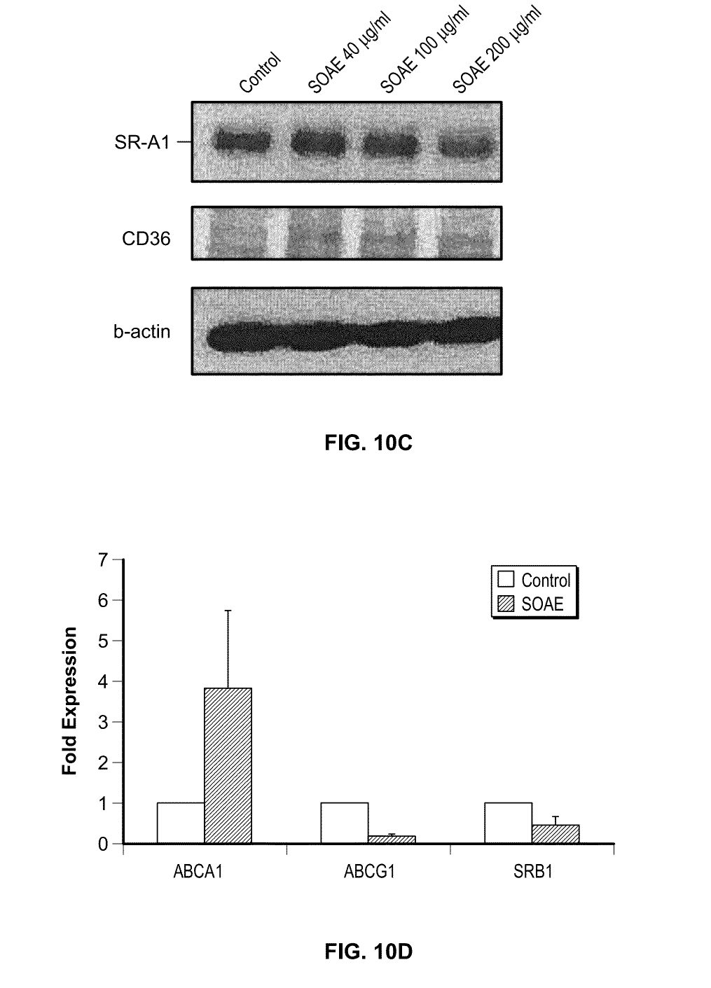

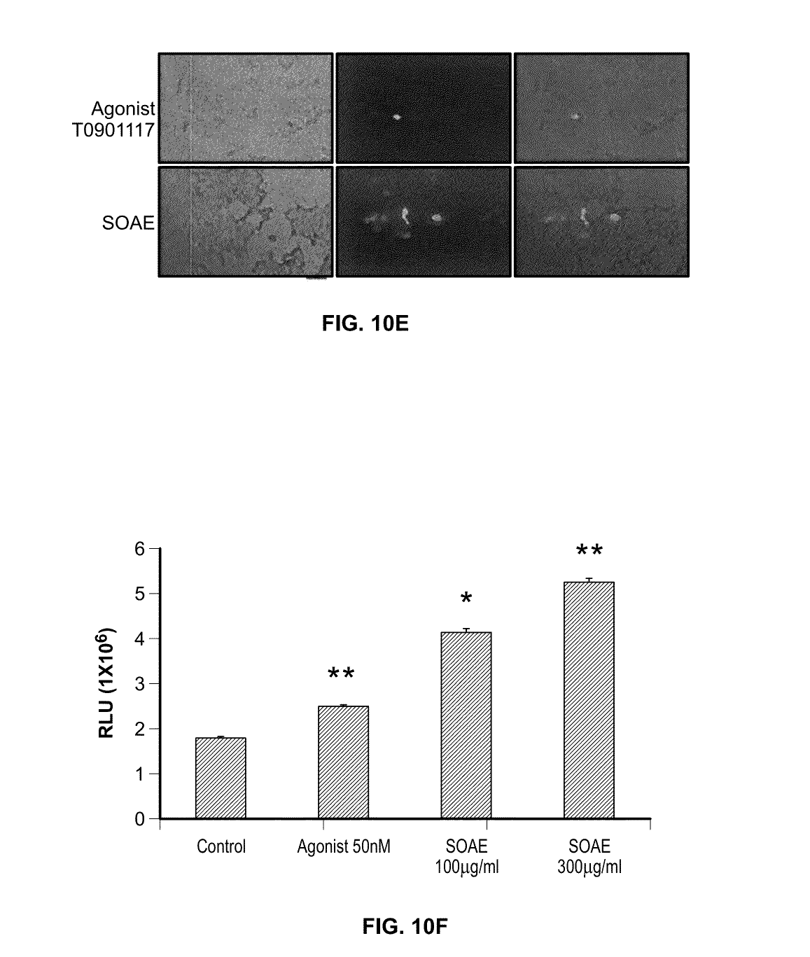

When HepG2-LXR reporter cell lines were incubated in the presence of SOAE, increased GFP expression (FIGS. 10A through 10F) was observed which indicated LXR activation. Increased luciferase activity was seen in the presence of SOAE (FIGS. 10A through 10F) thus confirming LXR activation by SOAE.

### Example 3

### PCR Array Analysis of Inflammatory Markers

RAW 264.7 cells (ATCC) were plated in 6-well plates at a density of 1×10⁶ cells per well and cultured in RPMI 1640 supplemented with 10% fetal bovine serum (FBS). The RAW cells were pre-treated with 500 μg/mL of SOAE for 2 hrs followed by addition of LPS (10 ng/mL). Cells were incubated for 24 hours. RNA was extracted using TRIZOL™ reagent after 24 hrs and used for atherosclerosis PCR array analysis (Qiagen). PCR array results showed that SOAE inhibited numerous inflammatory markers that were induced by LPS. The scatter plot (FIG. 5A) reveals the difference in expression pattern of numerous genes between LPS and LPS+SOAE groups. Genes such as Ccl2, Ccl5, IL-1α, IL-1β, TNF, Nfkb1, etc. were down-regulated in the presence of SOAE (FIG. 5B). Genes that were up-regulated in the presence of SOAE included ABCA1, Itgax, Plin2 and Serpine1 (FIG. 5C).

### Example 4

### Inhibition of LPS-Induced Inflammatory Cytokines in Mouse Peritoneal Macrophages by Sesame Seed Oil Aqueous Extract (SOAE)

Eight-week old Swiss Webster mice were purchased from Charles River, USA. All procedures were performed in accordance to the protocol approved by the Institutional Animal Care and Use Committee. Macrophages from the peritoneal cavity were isolated by peritoneal lavage using 3 mL of cold saline. Cells were plated in 6-well plates at a density of 1×10⁶ cells per well and were cultured in RPMI 1640 containing 10% fetal bovine serum at 37° C. in a 5% CO₂ incubator. The peritoneal macrophages were treated with LPS (10 ng/mL)±SOAE (200 μg/mL) for 24 hrs.

RNA was extracted using TRIZOL™ and gene expression of IL-1α, IL-6 and TNF-α was analyzed. Specifically, one μg of RNA was reverse transcribed into cDNA and quantitative real time PCR was performed with SYBR Green (Invitrogen, Carlsbad, Calif.). mRNA expression of inflammatory cytokines such as IL-1α, IL-6 and TNF-α was analyzed in macrophages.

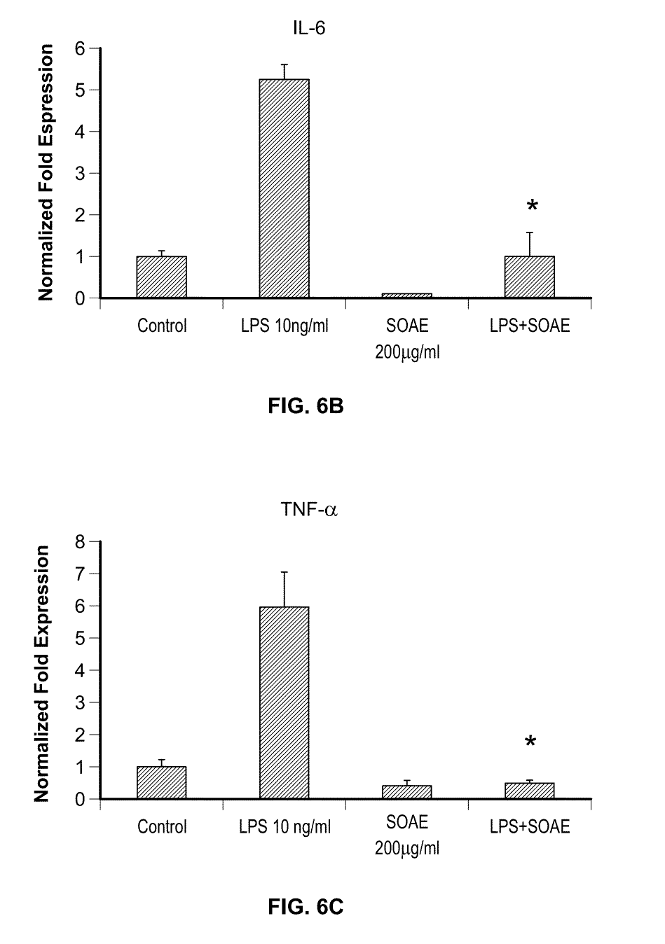

Similar results as seen with RAW macrophage cell line were observed. The cells responded well to the positive stimulus of 10 ng/mL of LPS as seen by significant induction of IL-1α, IL-6 and TNF-α. In the presence of SOAE, the mRNA expression of these inflammatory cytokines was significantly inhibited (FIGS. 6A through 6C).

### Example 5

### Inhibition of MCP1 and VCAM1 in HUVECs by Sesame Seed Oil Aqueous Extract (SOAE)

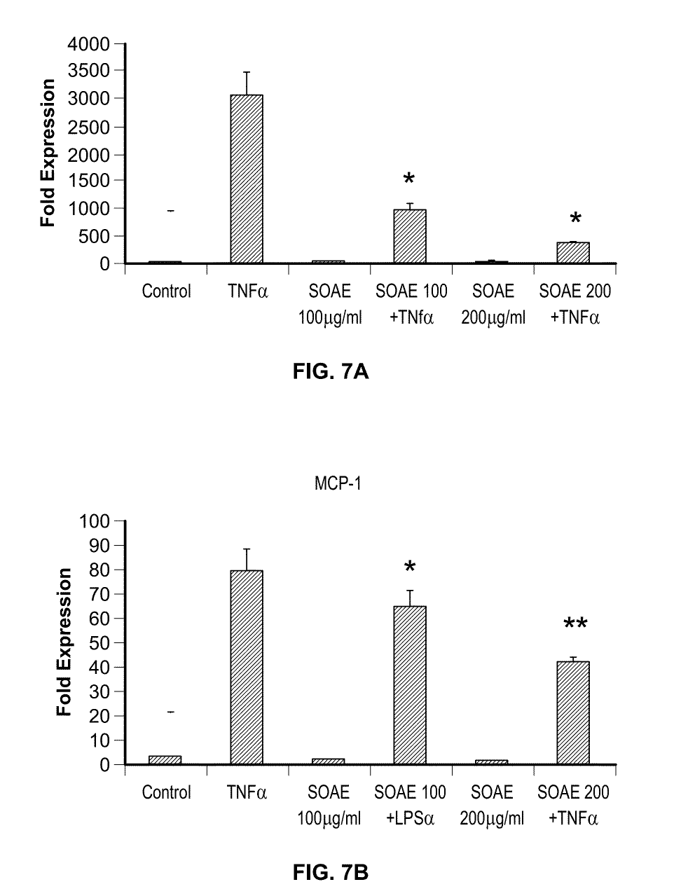

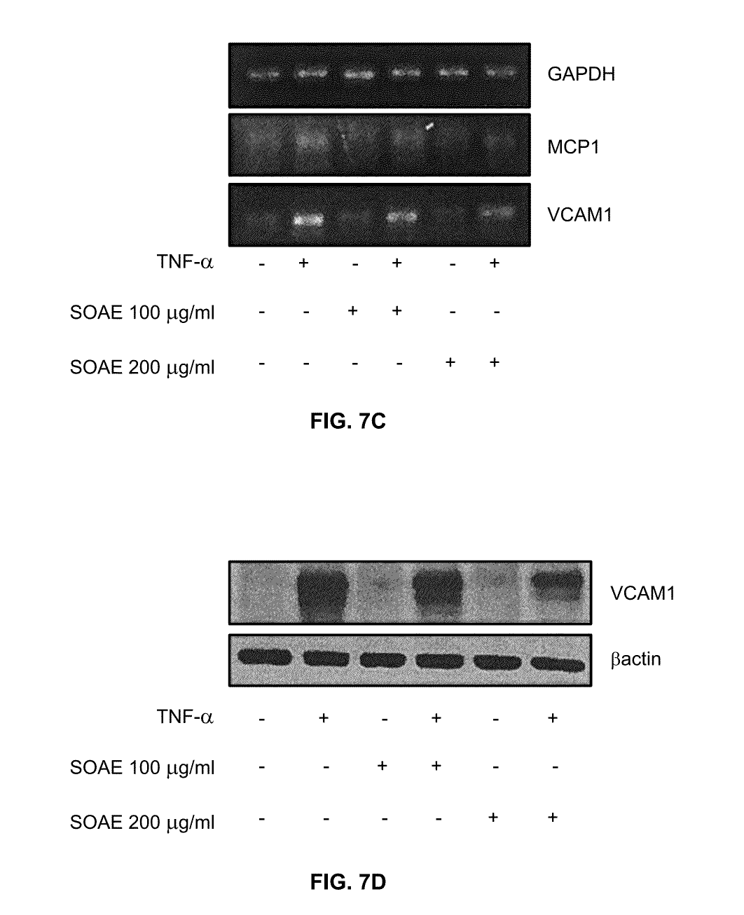

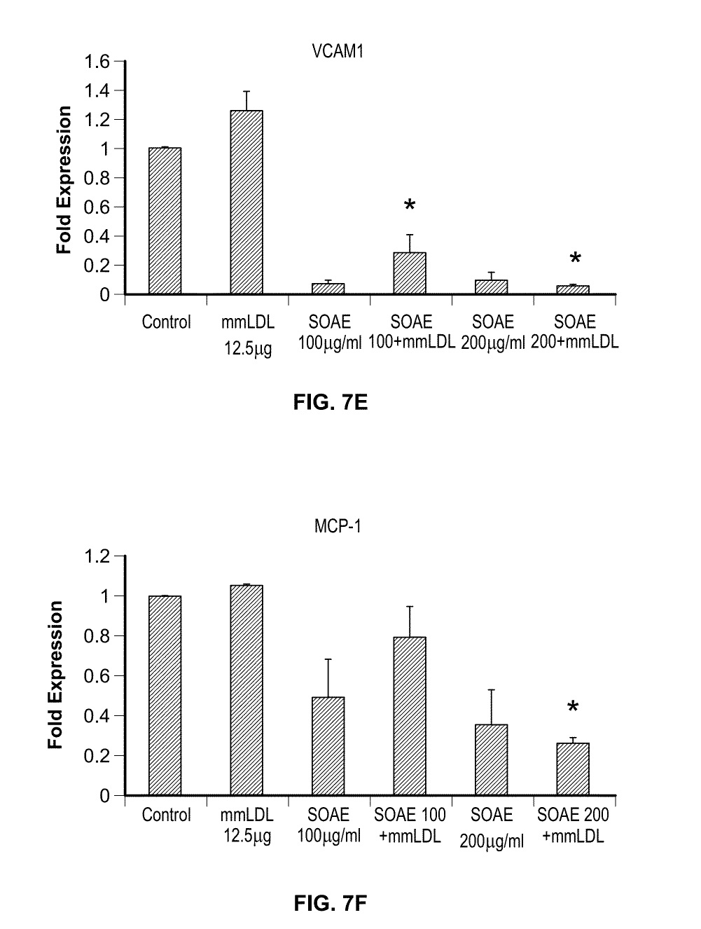

HepG2-LXR reporter cell line was obtained from System Biosciences and maintained in advanced DMEM with 10% FBS and 1 μg/mL puromycin. HUVECs were cultured in M199 with 20% FBS. HUVECs were treated with TNF-α (10 ng/mL)±SOAE (100 and 200 μg/mL). Cells were harvested after 24 hours and mRNA expression of VCAM1 and MCP-1 was analyzed. Specifically, RNA was extracted using TRIZOL™ reagent. One μg of RNA was reverse transcribed into cDNA and quantitative real time PCR was performed with SYBR Green (Invitrogen, Carlsbad, Calif.). mRNA expression of inflammatory cytokines such as IL-1α, IL-6 and TNF-α was analyzed in macrophages. VCAM1 and MCP-1 expression was analyzed in HUVECs. The primers for the genes are provided in FIG. 12. When cells were treated with TNF-α, strong induction of VCAM1 and MCP-1 mRNA expression was observed (FIGS. 7A through 7F). However, SOAE was able to inhibit both markers in a concentration dependent manner. FIGS. 7A through 7F show PCR products on agarose gel.

Total cellular protein was isolated from cells using RIPA lysis buffer. 15-25 μg of sample was fractionated on a 8-12% SDS polyacrylamide gel and transferred to a PVDF membrane for immunoblotting. Protein bands were visualized using ECL according to manufacturer's instructions. The Western blot for VCAM1 (FIGS. 7C and 7D) confirmed the results from real time PCR. As mm-LDL is more physiologically relevant in atherosclerosis, mm-LDL was used as another inducer of inflammation in HUVECs. SOAE was able to inhibit mm-LDL-induced VCAM1 and MCP-1 mRNA levels in endothelial cells (FIGS. 7A through 7F).

### Example 6

### Inhibition of NF-κB Transcription and Translocation in the Presence of Sesame Seed Oil Aqueous Extract (SOAE)

RAW 264.7 cells (ATCC) were cultured in RPMI 1640 supplemented with 10% fetal bovine serum (FBS). HepG2-LXR reporter cell line was obtained from System Biosciences and maintained in advanced DMEM with 10% FBS and 1 μg/mL puromycin. HUVECs were cultured in M199 with 20% FBS. Cells were cultured on sterilized glass cover slips in 6-well plates. They were treated with LPS (10 ng/mL) or TNF-α (10 ng/mL)±SOAE (200 μg/mL) for 24 hrs. The cells were then stained following supplier's protocol. The coverslips were mounted on glass slides and viewed at 100× under oil immersion using Zeiss fluorescence microscope.

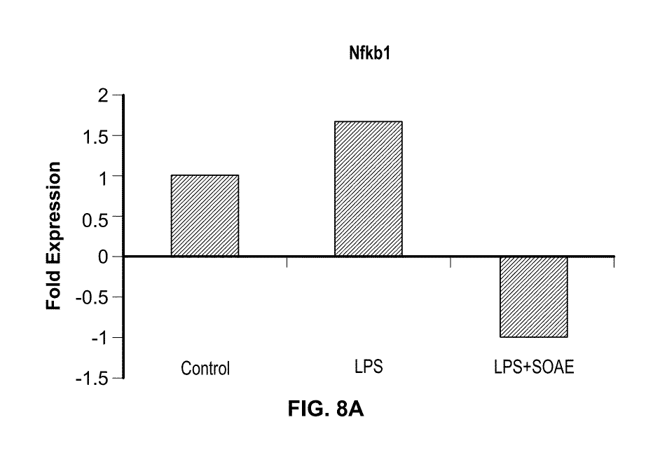

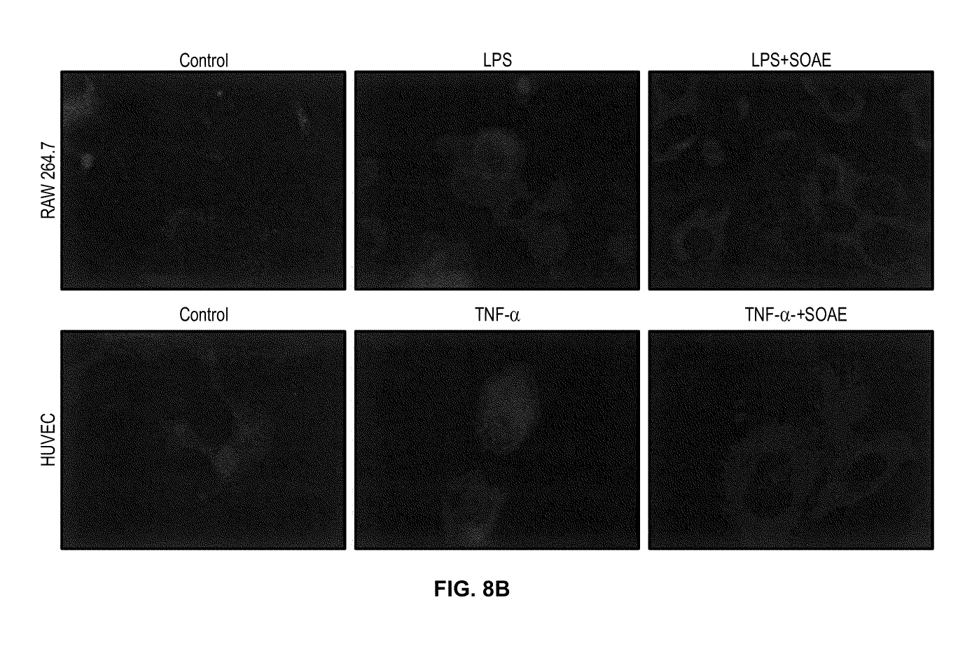

NF-κB is involved in LPS-induced expression of many of the inflammatory cytokines. PCR array results indicated suppression of NF-κB transcription in the presence of SOAE (FIG. 8A). The translocation of the transcription factor (p65) in the presence of SOAE was also studied. As shown in FIG. 8B, immunofluorescence staining of RAW cells and HUVECs showed reduced NF-κB translocation in to the nucleus thus attenuating LPS and TNF-α induced inflammation respectively.

### Example 7

### Inhibition of Lipoproteins by Sesame Seed Oil Aqueous Extract (SOAE)

Lipoproteins were isolated from normal plasma by sequential ultracentrifugation (Chung et al., Preparative and quantitative isolation of plasma lipoproteins: rapid, single discontinuous density gradient ultracentrifugation in a vertical rotor. J Lipid Res. 1980; 21: 284-291; Santanam et al., Paradoxical actions of antioxidants in the oxidation of low density lipoprotein by peroxidases. J Clin Invest. 95:2594-2600, 1995). They were subjected to oxidation with 5 μM copper or 0.2 U myeloperoxidase (MPO) with or without 100 μM tyrosine in the presence and absence of SOAE. The formation of conjugated dienes was monitored at an optical density of 234 nm. LDL oxidation was assessed by leucomethylene blue (LMB) assay and thiobarbituric acid reactive substances (TBARS). Minimally modified LDL (mm-LDL) was prepared as described previously (Chandrakala et al., Induction of brain natriuretic peptide and monocyte chemotactic protein-1 gene expression by oxidized low-density lipoprotein: relevance to ischemic heart failure. Am J Physiol Cell Physiol. 2012; 302(1):C165-77).

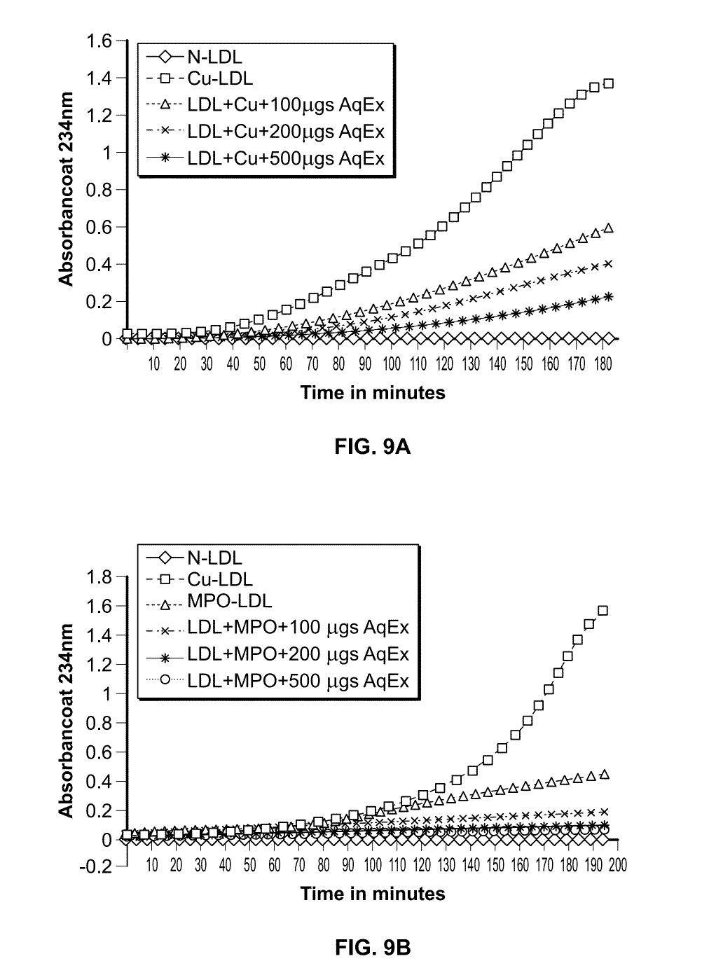

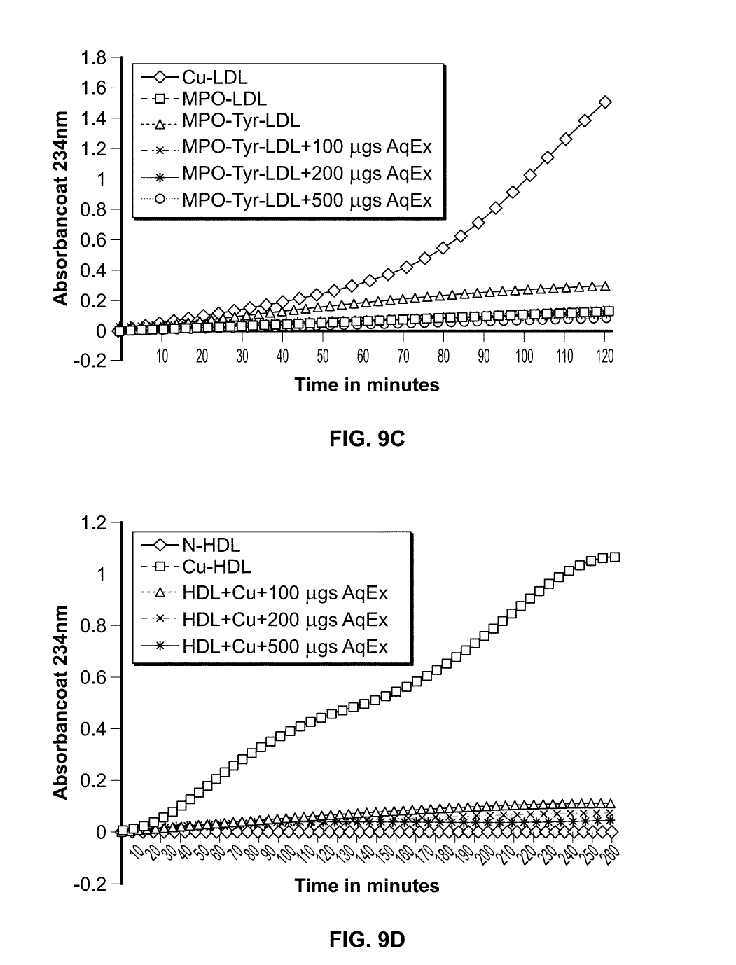

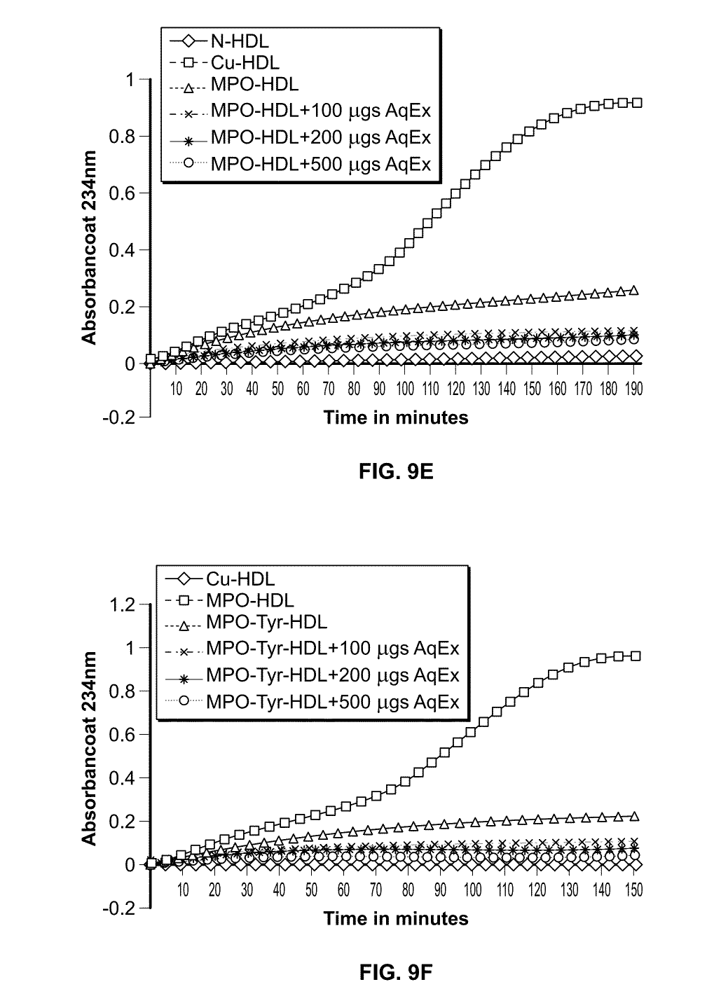

Oxidized form of LDL is proatherogenic as it is internalized by macrophages leading to cholesterol accumulation and foam cell formation (Steinberg. Low density lipoprotein oxidation and its pathobiological significance. J Biol Chem 1997; 272: 20963-20966). LDL was subjected to oxidation and the formation of conjugated dienes was measured by following increase in absorption at 234 nm. As shown in FIGS. 9A through 9F, in the presence of increasing amounts of SOAE there was an increase in lag time, suggesting that even low concentrations of SOAE were able to delay the oxidation rate. Similarly, SOAE inhibited the oxidation of LDL by MPO as well as by MPO and tyrosine (FIGS. 9A through 9F). LMB, TBARS assay, and electrophoretic mobility results also corroborated with the oxidation curves.

SOAE was also tested on HDL oxidation in presence and absence of 5 μM copper or 0.2 U MPO or MPO with tyrosine (FIGS. 9A through 9F). Similar results as with Ox-LDL were observed.

### Example 8

### Effect of Sesame Seed Oil Aqueous Extract (SOAE) on the Expression of Scavenger Receptors

Both class A scavenger receptor (SR-A1) and class B scavenger receptor (CD36) have been recognized for their involvement in atherogenesis (Babaev et al., Reduced atherosclerotic lesions in mice deficient for total or macrophage-specific expression of scavenger receptor-A. Arterioscler Thromb Vasc Biol. 2000; 20:2593-2599; Febbraio et al., Targeted disruption of the class B scavenger receptor CD36 protects against atherosclerotic lesion development in mice. J Clin Invest. 2000; 105:1049-1056). When RAW cells were treated with SOAE as detailed in the previous examples, the mRNA expression of scavenger receptor A1 (SR-A1) was inhibited (FIGS. 10A through 10F). However the mRNA levels of CD36 was significantly increased in a concentration dependent manner (FIGS. 10A through 10F). The protein expression of the scavenger receptors corroborated with the mRNA expression pattern as confirmed by western blot analysis (FIG. 10C).

### Example 9

### Effect of Sesame Seed Oil Aqueous Extract (SOAE) on Reverse Cholesterol Transport Genes

Reverse cholesterol transport is a mechanism that involves the transport of cholesterol from peripheral tissues to the liver for excretion. Increased RCT is desirable for atherosclerotic plaque regression. ABCA1, ABCG1 and SR-B1 are RCT genes known to be expressed by macrophages (Rader et al., The role of reverse cholesterol transport in animals and humans and relationship to atherosclerosis. J Lipid Res. 2009; 50 Suppl:S189-94). Both PCR array analysis and independent gene studies, as detailed in the previous examples, showed that SOAE significantly upregulated ABCA1 in RAW cells (FIGS. 5A through 5H, FIGS. 10A through 10F). However there was no significant effect on ABCG1 or SR-B1 (FIGS. 10A through 10F).

### Example 10

### In Vivo Effects of Sesame Seed Oil Aqueous Extract (SOAE) on LPS-Induced Inflammation

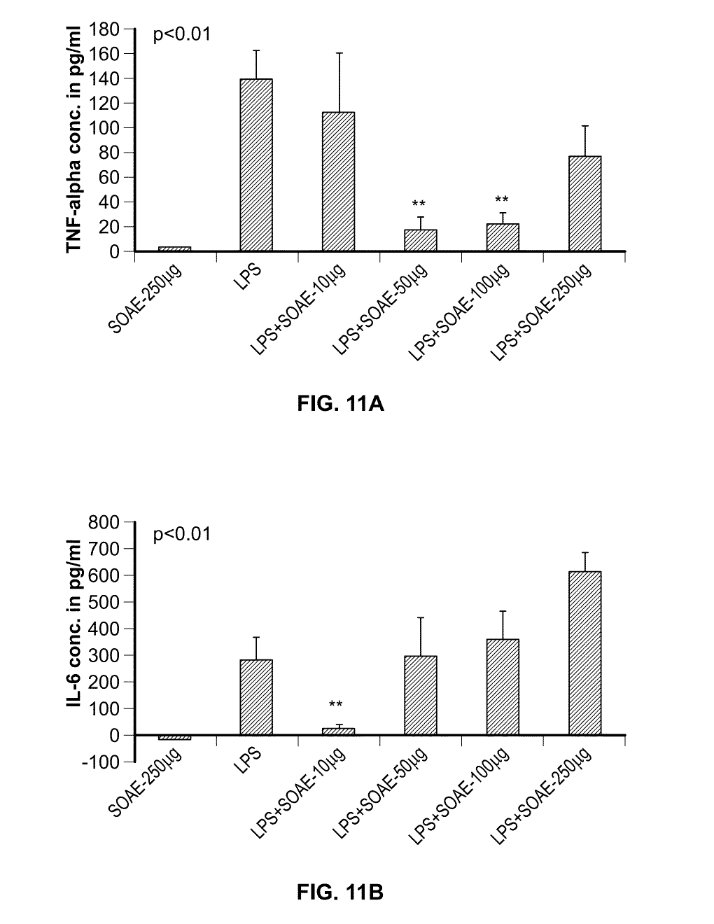

In order to determine whether SOAE would reduce LPS-induced inflammation in mice, animals were injected with LPS alone versus pretreatment with SOAE followed by LPS injection. Specifically, forty, six-week old female C57BL/6J mice (Jackson Laboratory, Bar Harbor, Me.) weighing approximately 20-22 g were divided into 6 groups: 12 animals in LPS group (6+6), 4 groups with 6 animals in each (LPS+SOAE), and 4 animals in control (SOAE 250 μg) group. To evaluate the effect of SOAE on LPS-induced inflammation, LPS (10 μg) and 10 μg LPS plus SOAE at varying concentration (10, 50, 100 and 250 μg/animal) were used. Animals were injected intraperitoneally at two different sites (200 μL/animal total injection volume) first with SOAE followed by LPS after half an hour. Blood and tissue samples were harvested from the animals 2 hrs after LPS injection. TNF-α and IL-6 cytokines were analyzed in mice serum samples using a sandwich ELISA kit (R & D systems, Minneapolis, Minn.) following manufacturer's protocol. As shown in FIG. 11A and FIG. 11B, pretreatment of mice with 50 μg and 100 μg SOAE (P<0.003) showed a significant reduction in LPS-induced TNFα levels as compared to LPS-treatment alone. A similar result was observed with IL-6 at 10 μg SOAE (P<0.004). However, a dose-dependent increase in IL-6 (FIG. 11A and FIG. 11B) levels was observed at concentrations higher than 10 μg SOAE.

Those skilled in the art will recognize, or be able to ascertain using no more than routine experimentation, many equivalents to the specific embodiments of the method and compositions described herein. Such equivalents are intended to be encompassed by the following claims.

It should be understood, of course, that the foregoing relates only to preferred embodiments of the present invention and that numerous modifications or alterations can be made therein without departing from the spirit and the scope of the invention as set forth in this disclosure. The present invention is further illustrated by the following examples, which are not to be construed in any way as imposing limitations upon the scope thereof. On the contrary, it is to be clearly understood that resort can be had to various other embodiments, modifications, and equivalents thereof which, after reading the description herein, can suggest themselves to those skilled in the art without departing from the spirit of the present invention and/or the scope of the appended claims.

What is claimed is:

1. A method of treating inflammation-related conditions, comprising, administering to a human or animal in need thereof an anti-inflammatory comestible composition comprising a food or beverage and a sesame seed oil aqueous extract, wherein the sesame seed oil aqueous extract is administered in an amount effective to reduce inflammation in the human or animal.

2. The method of claim 1, wherein the inflammation-related condition is selected from the group consisting of atherosclerosis, Alzheimer's Disease, allergies, myopathies, leukocyte defects, cancer, cardiovascular diseases, inflammatory bowel diseases, pelvic inflammatory diseases, and inflammatory diseases in the brain.

3. The method of claim 1, wherein the comestible composition is a beverage selected from the group consisting of coffee, tea, fruit juice, vegetable juice, milk, energy drink, protein drink, soft drink, and alcoholic drink.

4. The method of claim 1, wherein the comestible composition is a sweetener composition.

5. The method of claim 1, wherein the comestible composition further comprises a nutraceutical, pharmaceutical, or dietary supplement.

6. The method of claim 1, wherein the sesame seed oil aqueous extract is prepared by:
a) contacting a sesame seed oil with water to form a mixture;
b) mixing the mixture to form an aqueous layer and an oil layer; and
c) isolating the aqueous layer, which is substantially free of lipids, thereby producing the sesame seed oil aqueous extract.

7. The method of claim 6, further comprising dehydrating the sesame seed oil aqueous extract.

8. A method of preparing an anti-inflammatory comestible composition, comprising:
a) contacting a sesame seed oil with water to form a mixture;
b) mixing the mixture to form an aqueous layer and an oil layer;
c) isolating the aqueous layer, which is substantially free of lipids, to provide a sesame seed oil aqueous extract; and
d) combining the sesame seed oil aqueous extract with a food or beverage.

9. The method of claim 8, wherein mixing the mixture is by shaking the mixture, stirring the mixture, bubbling a gas through the mixture, or applying ultrasound to the mixture.

10. The method of claim 8, wherein isolating the aqueous layer is by filtration.

11. The method of claim 8, wherein from about 0.001 mg/mL to about 1000 mg/mL of the sesame seed oil aqueous extract is combined with the food or beverage.

12. The method of claim 8, further comprising dehydrating the sesame seed oil aqueous extract to form a powder before combining it with the food or beverage.

13. The method of claim 12, wherein from about 0.001 mg to about 1000 mg, w/w, of the powder is combined with the food or beverage.

14. The method of claim 8, wherein the beverage is selected from the group consisting of coffee, tea, fruit juice, vegetable juice, milk, energy drink, protein drink, soft drink, and alcoholic drink.

15. An anti-inflammatory comestible composition, comprising: a food or beverage and a sesame seed oil aqueous extract in an amount effective to reduce inflammation in a human or animal.

16. The composition of claim 15, wherein the sesame seed oil aqueous extract is prepared by:
a) contacting a sesame seed oil with water to form a mixture;
b) mixing the mixture to form an aqueous layer and an oil layer; and
c) isolating the aqueous layer, which is substantially free of lipids, thereby producing the sesame seed oil aqueous extract.

17. The composition of claim 16, wherein the sesame seed oil aqueous extract is present in the composition at from about 0.001 mg/mL to about 1000 mg/mL.

18. The composition of claim 16, wherein the aqueous layer is dehydrated to form a powder.

19. The composition of claim 18, wherein from about 0.001 mg to about 1000 mg, w/w, of the powder is combined with the food or beverage.

20. The composition of claim 15, wherein the composition is a beverage selected from the group consisting of coffee, tea, fruit juice, vegetable juice, milk, energy drink, protein drink, soft drink, and alcoholic drink.
